(19) United States
(12) Patent Application Publication
(10) Pub. No.: US 2008/0124286 A1
(43) Pub. Date: May 29, 2008

(54) Algae supplement and treatment method

(76) Inventor: Jerold B. Lisson, Henrietta, NY (US)

Correspondence Address: LAW OFFICE OF TRACY P. JONG, 2775 BUFFALO ROAD, SUITE 3, ROCHESTER, NY, 14624, US

(21) Appl. No.: 11/893,244

(22) Filed: Aug. 15, 2007

Related U.S. Application Data
(63) Continuation-in-part of application No. 11/800,018, filed on May 3, 2007.
(60) Provisional application No. 60/861,462, filed on Nov. 28, 2006.
(60) Provisional application No. 60/903,068, filed on Feb. 23, 2007.

(51) Int. Cl. A61K 8/00; A61K 36/02; A61P 43/00; A61K 48/00

(52) U.S. Cl. 424/61; 424/195.17; 424/70.1; 424/93.4

(57) ABSTRACT

This invention relates to a method and composition for enhancing epidermal and/or hair follicle stem cell production, cell renewal, and/or growth of the skin, hair and nails, by topical administration of a therapeutic dosage of cyanobacteria and green algae and/or both simultaneous enteral and topical administration of a therapeutic dosage of cyanobacteria and green algae.

### PRIORITY CLAIM AND RELATED APPLICATIONS

This continuation-in-part application claims the benefit of priority from U.S. Ser. No. 11/800,018 filed on May 3, 2007. This application also claims the benefit of priority from provisional application U.S. Ser. No. 60/861,462 filed on Nov. 28, 2006 and provisional application U.S. Ser. No. 60/903,068 filed on Feb. 23, 2007 in the name of Jerold Lisson of 61 Blackwell Lane, Henrietta, N.Y. Each of said applications is incorporated by reference in its entirety.

### FIELD

This invention relates to a method and composition for enhancing epidermal and hair follicle stem cell production, cell renewal, and/or growth of the skin, hair and nails, by topical administration of a therapeutic dosage of cyanobacteria and green algae and/or both simultaneous enteral and topical administration of a therapeutic dosage of cyanobacteria and green algae.

### BACKGROUND

Two concomitant factors that affect the health or well being of higher ordered animals, such as humans and other mammals, are the ability to regenerate damaged tissue and the prevention of damage to tissues. It has been observed that some primitive cultures who supplement their diet with green algae appear to be healthier and experience longer life spans than western civilizations. They observed a much speedier recovery from illnesses, faster healing of wounds, and an apparent slower aging process. Researchers acknowledge much of this may be due to socio-psycho influences on their life style and the very limited exposure to western diseases. However, they also observed many differences in their dietary habits. One notable difference was the use of cyanobacteria and green algae in their diet.

The benefits of algae are also realized when administered topically. This may be a stand-alone treatment, or combined with enteral administration of algae. The benefits are amplified when the combined enteral and topical treatment method is employed.

Research has shown that the cyanobacteria produces and stores large quantities of chemicals in the L-selectin ligand family. These and other component chemicals are known to play a key role in slowing or ceasing the aging process of pluripotent cells. They play a role in the expression of human genes which are mediated by pluripotent stem cells. Thus, Applicant believes that the use of specific species of cyanobacteria and/or green algae (or their respective extracts) increase the lifetime and activity of available pluripotent cells via antioxidant protective mechanisms available from the algae combinations of the novel composition. This, in turn, allows for the faster replacement of damaged tissue and consequently reduces the risk of pathogens colonizing on damaged tissue and subsequent return to healthy function. Organs and tissues remain healthier as the replacement of aged cells is quicker with the increased pluripotent cell availability, slowing down the natural aging process. Reference may be had to, e.g., “Nutraceuticals Promote Proliferation of Human Stem Cells” by Paula Bickford, et al., published in Stem Cells and Development vol. 15 (2006) and “Blueberry- and spirulina-enriched diets enhance strital dopamine recovery and induce a rapid, transient microglia activation after injury of the rat nigrostriatal dopamine system” by Ingrid Stromberg, et al., published in Experimental Neurology 196 (2005) 298-307.

U.S. Pat. No. 6,814,961 discloses a method to enhance trafficking or homing of stem cells by administering a therapeutically effective amount of blue-green algae to an animal subject. The compounds described therein are effective, however, do not contemplate the synergistic effects of additional potent antioxidant components that increase the activity of and prolong the life cycle of the pluripotent cells. Additionally, Inventor-Applicant believes that his novel composition incorporates a greater concentration of pluripotent cell enhancing cyanobacteria than is commercially available in supplement form.

The cyanobacteria and green algae are a remarkable storehouse of major nutrients that are synergistic in their natural form. The novel algae composition of this invention has extraordinary regenerative and prophylactic power via an optimal mix of two to four inter-supporting and synergistic green algae and cyanobacteria components. This interaction eliminates the need for multiple food sources currently consumed in the average diet. Therapeutic action of astaxanathin of the green algae includes protective antioxidant activity and inhibition of the catabolic effects of heavy exercise by muscle tissue cellular oxidation. Applicant believes the simultaneous synergy of the potent antioxidant action and pluripotent cell production provides an improvement over the prior art.

Similarly, Applicant believes that bioorganisms will be more receptive to, and benefit more from, the regeneration processes offered by the additional pluripotent cell production. By combining cyanobacteria with green algae components, potent antioxidant activity enhances the general health of the subject and inhibits the harmful effects of disease causing organisms, thus the regenerative process benefits will be amplified.

Stem cells are pluripotent cells derived from stromal or basal tissue capable of differentiating into more specialized cells. Stem cells, from both stromal and basal tissue, have been found to differentiate into a variety of non-hematopoietic tissue specific cell types, such as myocytes, heptocytes, osteocytes, glial cells, epithelial cells and neurons. Hematopoietic stem cells can also differentiate into many different types of blood cells including red blood cells, platelets and leukocytes. Hematopoietic stem cells are quite abundant and play a role in the continuous lifelong replenishment of blood cells.

U.S. Pat. No. 6,814,961 discloses that a therapeutically effective amount of blue-green algae induces a transient increase in the population of some stem cells, such as CD34+ stem cells, in the subject's circulatory system. More specifically, the administration of a polysaccharide rich fraction of whole AFA increases the homing of CD34+ and NK cells from the circulatory system to various parts of the body. The entire disclosure of said patent is incorporated by reference herein. The percentage increase in the number of circulating stem cells, compared to a control, is ranges from about 10% to more than about 500%, following administration of the blue-green algae. U.S. Pat. No. 6,814,961 further discloses studies that determined the circulating levels of CD34+ stem cells decreased to near pre-existing levels within 4 hours.

The prophylactic use of natural antioxidants to increase the lifetime of cells in mammals has been well recognized. The antioxidants react with toxins (free radicals) either in the circulatory system or at cell site to inactivate the toxins prior to damaging the cells. U.S. Pat. No. 7,074,990 describes the role of specific carotenoids in the lifetime extension of pluripotent cells. U.S. Pat. No. 6,884,783 describes the use of other antioxidants such as 7-hydroxy chromones for enhancing the lifetime of pluripotent cells. U.S. Pat. No. 7,025,965 describes the use of phycocyanins from blue green algae for the treatment of inflammation by reacting with toxins causing cell destruction. The entire disclosure of each of said patents is incorporated by reference herein.

Algae is rich in many antioxidants such as beta-carotene, astaxanthin, phycocyanins, 7-hydroxy chromones and a plurality of unspecified antioxidants. Some of these antioxidants comprise polysaccharides and components thereof. Many of these antioxidants are specific to certain pluripotent cells (e.g. 7-hydroxy chromones).

The synergy between the pluripotent cell enhancing components and the antioxidant components yields unexpected results that are more than the benefits of the sum of each component individually. Secondly, the paucity of ingredients provides for a nutritional supplement that is both simpler and lower cost to manufacture. Additionally, with fewer ingredients, there is less likelihood of interactions among the various components of the composition and less likelihood of negative reactions (e.g., drug interactions, allergic reactions, side effects, and the like) for the subject. Not only are there fewer components, but the prophylactic, therapeutic and regenerative properties of the components are superior to those utilized in prior art supplements and allow for custom blending of the components as medically indicated or desirable for each individual subject.

The Applicant has found that a novel algae composition comprising a liquid frozen or powdered form of whole plant extracts of cyanobacteria and at least one green algae as active components provides enhanced stamina and endurance in motor function, for example, by prolonging the ability to perform repetitive and/or high stress physical movements by delaying the onset of muscle fatigue and/or minimizing the degrading effects. The novel algae composition also improves general health physically and neurologically.

The Applicant has also found that a novel algae composition comprising a powdered form and or frozen liquid form of whole plant extracts of cyanobacteria and at least one green algae as active components provides enhancement of additional processes—those commonly known as epidermal cell renewal. When applied to the skin, a simple solution, paste or cosmetic preparation containing a composition of both cyanobacteria and green algae has both an alimentary and a beneficial cosmetic function by directly stimulating basal cell division.

The Applicant has also found that the topical application (to the nails and nail beds) of a novel algae composition comprising a powdered form and or frozen liquid form of whole plant extracts of cyanobacteria and at least one green algae as active components increases the rate of nail growth by at least 200%.

Inventor-Applicant believes that his novel composition incorporates a unique combination of microbial species and a greater concentration of pluripotent cell enhancing cyanobacteria and green algae than is commercially available in topical form.

Thus, it is desirable to provide a compound and treatment method that enhances pluripotent cell (e.g., bone marrow and epidermal stem cell) production, enhances pluripotent cell emission into the bloodstream, increases the activity of the pluripotent cells, prolongs the life cycle of the pluripotent cells, enhances the homing of the pluripotent cells to damaged or stressed tissues, and optimizes the general physiological condition of the subject so the beneficial regenerative effects can be realized by absorption in said stressed tissues.

Thus, it is also desirable to provide a compound and treatment method that enhances epidermal stem cell production, stimulates basal cell division, increases the activity of the epidermal stem cells, and prolongs the life cycle of the epidermal stem cells. The novel compositions of this invention are advantageous over prior art topical cosmetic and healing compositions. The synergy between the epidermal stem cell enhancing components and the antioxidant components yield unexpected results that are more than the benefits of the sum of each component individually.

Additionally, the synergy between the topical and enteral administration of the novel algae compositions yields unexpected results that are more than the benefits of the sum of each component individually. The novel algae compositions and treatment methods of this invention have superlative regenerative and prophylactic power due to the inherent and synergistic properties of the green algae and cyanobacteria components.

### SUMMARY

In accordance with this invention, there is provided a composition comprising a therapeutic dosage of green algae and a therapeutic dosage of cyanobacteria, wherein the composition is formulated for topical application. In one embodiment, said green algae comprises a green algae selected from the group consisting of Haemotococcus pluvialis, Chlorella vulgaris, Chlorella pyrenoidosa, Dunaliella salina or a combination thereof. In one embodiment, said cyanobacteria comprises a cyanobacteria selected from the group consisting of Aphanizomenon flos-aquae, Spirulina pacifica, Spirulina plantesis or a combination thereof.

The green algae and cyanobacteria contain certain chemical growth factors that enhance pluripotent and stem cell growth. The green algae and cyanobacteria also contain certain antioxidants that protect cells against damage.

In accordance with this invention, there is also provided a method for enhancing epidermal stem cell growth in a subject, comprising: administering to a subject a topical composition comprising a therapeutic dosage of green algae and cyanobacteria. In one embodiment, the therapeutic administration of the novel composition of this invention induces the increase of epidermal stem cell growth by as much as 25%, by as much as 50%, by as much as 75%, by as much as 100%, by as much as 150%, by as much as 200%, by as much as 250%, by as much as 300%, by as much as 400%, and by as much as 500%.

In accordance with this invention, there is also provided a method for increasing the longevity (i.e. lifetime) of epidermal stem cells in a subject, comprising: administering to a subject a topical composition according to this invention. In one embodiment, the antioxidant activity in the novel composition of this invention induces the longevity of epidermal stem cells by more than about 4 hours, by more than about 6 hours, by more than about 8 hours, by more than about 12 hours, by more than about 24 hours, by more than about 36 hours, by more than about 48 hours, and by more than about 72 hours. This can dramatically increase the effectiveness of the epidermal stem cells in replicating and/or treating disease both at normal levels and the increased levels during the transient period.

In accordance with this invention, there is also provided a method for enhancing a rate of nail growth in a subject, comprising: administering to a subject a topical composition according to this invention and an enteral composition comprising a therapeutic dosage of green algae and a therapeutic dosage of cyanobacteria. In one embodiment, the therapeutic administration of the novel composition of this invention induces an increase in the rate of nail growth by as much as 200%.

In accordance with this invention, there is also provided a method for increasing a rate of proliferation of epidermal cells in a subject, comprising: administering to a subject a topical composition according to this invention. In one embodiment, the therapeutic administration of the novel composition of this invention induces an increase in the rate of epidermal cell proliferation by as much as 200%.

In accordance with this invention, there is also provided a method for increasing a rate of proliferation of hair follicle stem cells in a subject, comprising: administering to a subject a topical composition according to this invention. In one embodiment, the therapeutic administration of the novel composition of this invention induces an increase in the rate of hair follicle stem cell proliferation by as much as 200%.

In accordance with this invention, there is also provided a method for increasing the longevity (i.e. lifetime) of hair follicle stem cells in a subject, comprising: administering to a subject a topical composition according to this invention. In one embodiment, the antioxidant activity in the novel composition of this invention induces the longevity of hair follicle stem cells by more than about 4 hours, by more than about 6 hours, by more than about 8 hours, by more than about 12 hours, by more than about 24 hours, by more than about 36 hours, by more than about 48 hours, and by more than about 72 hours. This can dramatically increase the effectiveness of the hair follicle stem cells in replicating and/or treating hair transplantation.

In accordance with this invention, there is also provided a method for increasing a rate of proliferation of epidermal stem cells in a subject, comprising: administering to a subject a topical composition according to this invention and administration of an enteral composition comprising a therapeutic dosage of green algae and a therapeutic dosage of cyanobacteria.

The compositions and methods of this invention enhance the skin's visual appearance and protect the skin against development of wrinkles, loss of firmness, loss of elasticity, discoloration, loss of smooth surface texture, undesirable pigmentation, reduction in cell replication rates, reduction in skin's ability to repair itself and other problems with structure and/or function. The cosmetic compositions according to the invention are also useful as anti-aging compositions for improving the quality and appearance of the skin.

An enteral composition is disclosed herein for enhancing health and disease control in humans, comprising: a therapeutic dosage comprising green algae and cyanobacteria. In one embodiment, said green algae comprises a green algae selected from the group consisting of Haemotococcus pluvialis, Chlorella vulgaris, Chlorella pyrenoidosa, Dunaliella salina or a combination thereof. In one embodiment, said cyanobacteria comprises a cyanobacteria selected from the group consisting of Aphanizomenon flos-aquae, Spirulina pacifica, Spirulina plantesis or a combination thereof.

The novel algae composition of this invention provides therapeutic and prophylactic health benefits in the nature of protection of cellular tissues from the degenerative processes of free radicals and aging.

In some embodiments, the subject provided the treatment is healthy. In other embodiments the subject has known disease or physiological disorders.

The novel algae composition of this invention may be topically administered therapeutically or prophylactically, or most preferably, in a therapeutic and prophylactic combination. The novel topical composition may be administered in combination with an enteral composition comprising a therapeutic dosage of green algae and a therapeutic dosage of cyanobacteria. The treatment is most effectively delivered in multiple doses at regular intervals.

It is an object of this invention to provide an algae composition which enhances epidermal stem cell production.

It is an object of this invention to provide an algae composition and method which enhances the life cycle of epidermal stem cells.

It is an object of this invention to provide an algae composition and method which enhances epidermal cell proliferation.

It is an object of this invention to provide an algae composition and method which enhances the rate of basal cell division.

It is an object of this invention to provide an algae composition and method which increases the rate of nail growth.

In some embodiments, the subject provided the treatment is healthy. In other embodiments the subject has known disease or physiological disorders.

It is yet another object of this invention to provide a novel algae composition that is economical for mass production from the viewpoint of the manufacturer and consumer, thereby making it economically available to the buying public.

Whereas there may be many embodiments of the present invention, each embodiment may meet one or more of the foregoing recited objects in any combination. It is not intended that each embodiment will necessarily meet each objective.

Thus, having broadly outlined the more important features of the present invention in order that the detailed description thereof may be better understood, and that the present contribution to the art may be better appreciated, there are, of course, additional features of the present invention that will be described herein and will form a part of the subject matter of the invention.

In this respect, before explaining at least one embodiment of the invention in detail, it is to be understood that the invention is not limited in its application to the details of construction and the arrangements of the components set forth in the following description. The present invention is capable of other embodiments and of being practiced and carried out in various ways. Also it is to be understood that the phraseology and terminology employed herein are for the purpose of description and should not be regarded as limiting.

### BRIEF DESCRIPTION OF THE DRAWINGS

The invention will be described by reference to the specification and the drawings, in which like numerals refer to like elements, and wherein:

FIG. 1 is a perspective view of a fingernail following treatment according to a novel method of this invention;

FIG. 2 is a photograph that depicts the right hand after about 8 weeks of treatment;

FIG. 3 is a photograph that depicts the left hand after about 8 weeks of treatment;

FIG. 4 is a photograph that depicts the thumb of the right hand after about 8 weeks of treatment;

FIG. 5 is a photograph that depicts the index finger of the right hand after about 8 weeks of treatment;

FIG. 6 is a photograph that depicts the index finger of the right hand after about 2 weeks of treatment;

FIG. 7 is a photograph that depicts the ring finger of the right hand after about 2 weeks of treatment;

FIGS. 8 and 9 are photographs that depict the middle finger of the right hand after about 2 weeks of treatment;

FIG. 10 is a photograph that depicts the thumb of the right hand after treatment.

The drawings are not to scale, in fact, some aspects have been emphasized for a better illustration and understanding of the written description.

The patent or application file contains at least one photograph executed in color. Copies of this patent with color drawings/photographs will be provided by the Office upon request and payment of the necessary fee.

### DEFINITIONS

As used in this specification, stem cell means a pluripotent, self-maintaining cell that gives rise to progeny of many tissue types, including but not limited to the entire hematopoietic and bone marrow stromal cell lineages. A typical endogenous stem cell resides in the bone marrow, although stem cells also reside in the intestinal epithelium, tongue mucosa, epidermis, testis, and the like. It is to be understood that the invention is described with reference to primarily bone marrow stem cells, but that it is intended to apply to these other stem cell types as well.

As used in this specification, cyanobacteria means a gram-negative photosynthetic bacteria belonging to Division Cyanophyta that may exist in unicellular, colonial, or filamentous forms. By way of example, but not limitation, cyanobacteria may include, but are not limited to, Spirulina species, aphanizomenon species, Aphanizomenon flos aquae (AFA), and the like. In a preferred embodiment, cyanobacteria comprises an L-selectin ligand. Cyanobacteria may also be known as blue-green algae.

As used in this specification, green algae means photosynthetic paraphyletic organism, a eukaryote with organelles that may exist in unicellular, diatomic, colonial, or filamentous forms. By way of example, but not limitation, green algae may include, but are not limited to, Chlorella species (e.g., Chlorella vulgaris, Chlorella pyrenoidosa and the like), Haematoccocus pluvialis (H. pluvialis) species, Dunaliella species, and the like. In a preferred embodiment, green algae comprises astaxanathin.

As used in this specification, algae means any fraction, extract, or isolated or purified molecule from an algae cell. In one embodiment, the component is a protein or nucleic acid. In another embodiment, the component is a phytochemical. In another embodiment, the component is a fraction of algae. Extracts may be prepared according to the teachings of U.S. Pat. Nos. 6,977,076 (methods for obtaining from Chlorella extract polysaccharides having immunomodulatory properties), 6,814,961 (method for enhancing stem cell trafficking), and the like.

As used in this specification, cyanobacteria means any fraction, extract, or isolated or purified molecule from an cyanobacteria cell. In one embodiment, the component is a protein or nucleic acid. In another embodiment, the component is a phytochemical. In another embodiment, the component is a fraction of cyanobacteria. Extracts may be prepared according to the teachings of U.S. Pat. Nos. 6,977,076 (methods for obtaining from Chlorella extract polysaccharides having immunomodulatory properties), 6,814,961 (method for enhancing stem cell trafficking), and the like.

As used in this specification, Spirulina refers to Arthrospira (Spirulina) platensis and/or Spirulina pacifica.

As used in this specification, Chlorella refers to a genus of water-grown unicellular green algae belonging to the Phylum Chlorophyta.

As used in this specification, effective amount means an amount of compound or mixture capable of activating or enhancing pluripotent cell production that can be determined by various methods used in the biological sciences, including generating an empirical dose-response curve. In one embodiment, a “therapeutically effective amount” is an amount effective for enhancing production of pluripotent cells that replenish, repair, or rejuvenate tissue. A therapeutically effective amount also may be an amount sufficient for treating a condition or disease.

As used in this specification, administration to a subject means oral or parenteral administration of the novel compound in the form of a unit dose in solid, semi-solid, or liquid dosage form such as tablets, pills, powders, gels, pastes, capsules, coated tablets, liquid solutions, or liquid suspensions.

As used in this specification, tissue means any organized mammalian cell structure, such as, but not inclusive of epithelium, muscle, liver, lung or bone.

As used in this specification, disease management means the destruction of non organized pathogens (destructive bacteria, parasites, or viruses) as they enter the human system via open wounds, ingestion, breathing and the like and the destruction of colonized pathogens in the form of wound abscesses, pneumonia, intestinal infections, and the like. These colonized pathogens may be in any tissue or body fluid and may be caused by an undefined species of pathogens.

As used in this application, a toxin means any chemical that either reacts with the plasma membrane or cytoplasm to prevent cell respiration or metabolism. By way of illustration, but not limitation, toxins may be oxidants or free radical generators.

As used in this application, immunoglobulin means a polysaccharide on the membrane surface or in the fluidic system of a cell whose function is to intercept and eliminate toxins.

As used in this application, active ingredient is any component comprising either a chemical moiety, cells or cell extract that is necessary for the therapeutic regime of a subject.

As used in this application, inert ingredient means any non-therapeutic material added to the mixture which does not contribute to the generation, homing or lifetime enhancement of pluripotent cells and may include, but is not limited to, flavorings, sweeteners, surfactants, solid bulk diluents, binders, pharmaceutically acceptable carriers, texture enhancers, palatability enhancers, preservatives and the like.

As used in this application, pharmaceutically acceptable carrier means any bulk medium that the algae and cyanobacteria are add to for the proper administration of the algae and cyanobacteria. They may include water, lipids and/or. These also may contain adjunct materials such as salts, surfactants, buffers, thickeners and preservatives.

As used in this application, texture enhancer means any bulk diluent added to the mixture to improve consistency such that it may be administered orally to the subject.

As used in this application, palatability enhancer means materials added to the mixture to make oral administering of the algae and cyanobacteria more pleasant to the subject. These may include flavorings and sweeteners.

As used in this application, circulatory system means the structure that moves blood and blood components throughout the body of animals, including the heart, blood vessels and lymph vessels.

As used in this application, diseased site means a site in an animal where nonconforming cells are present. These nonconforming cells may either be pathogens, such as bacteria and viruses, or abnormal cells such as carcinomas.

As used in this application, injured site means any tissue that is damaged either by mechanical, chemical or bacterial means.

As used in this application, enhanced concentration site means any site where the concentration of pluripotent cells is elevated above the concentration measured at normal conditions.

As used in this application, antioxidant means any chemical moiety that reacts with chemicals containing free radicals, reducing their capability to react/oxidize bioactive chemicals in cells.

As used in this application, pluripotent cells are nondifferentiated stem or daughter cells whose progeny produce specific tissues such as blood, bone, neuron, epithelial and the like.

As used in this application, liquid suspension is any uniform mixture consisting of a liquid containing undissolved solids.

As used in this application, beta-CDS is the scientific identifier for a super family of Immunoglobulins.

As used in this application, environmental toxin is any water or airborne chemical or biological moiety which will cause negative cell function.

As used in this application, tablet means a solid form single dosage such as gel caplet, pill, capsule, flake or the like.

As used in this application, proliferation means rapid and repeated production of new parts such as in a mass of cells by a rapid succession of cell divisions.

### DETAILED DESCRIPTION

The present invention concerns a method and composition for enhancing skin health and skin disease control in a subject. In some embodiments, the subject provided the novel algae composition is healthy. In other embodiments, the subject suffers a disease or physiological condition. In some embodiments, the subject is a human subject. In other embodiments, the subject is a veterinary subject, preferably a multicellular, vertebrate organism including, for example, mammals.

In one embodiment of the novel composition of this invention, the composition comprises a therapeutic dosage of green algae and a therapeutic dosage of cyanobacteria and the composition is formulated for topical application. In accordance with this invention, there is also provided a method for increasing the longevity of epidermal stem cells in a subject, comprising: administering to a subject a topical composition according to this invention.

In one embodiment of the novel composition and treatment method of the present invention, said green algae comprises a green algae selected from the group consisting of Haemotococcus pluvialis, Chlorella vulgaris, Chlorella pyrenoidosa, Dunaliella salina or a combination thereof. In one embodiment, said cyanobacteria comprises a cyanobacteria selected from the group consisting of Aphanizomenon flos-aquae, Spirulina pacifica, Spirulina plantesis or a combination thereof.

In one embodiment of the novel composition and treatment method of the present invention, said green algae comprises at least two green algae selected from the group consisting of Haemotococcus pluvialis, Chlorella vulgaris, Chlorella pyrenoidosa, and Dunaliella salina.

In one embodiment of the novel composition and treatment method of the present invention, said cyanobacteria comprises at least two cyanobacteria selected from the group consisting of Aphanizomenon flos-aquae, Spirulina pacifica, and Spirulina plantesis.

In one embodiment of the novel composition of this invention, the topical composition further comprises xylitol.

In one embodiment, the antioxidant activity in the novel topical composition of this invention induces the longevity of epidermal stem cells by more than about 4 hours, by more than about 6 hours, by more than about 8 hours, by more than about 12 hours, by more than about 24 hours, by more than about 36 hours, by more than about 48 hours, and by more than about 72 hours. This can dramatically increase the effectiveness of the epidermal stem cells in treating disease both at normal levels and the increased levels during the transient period.

In one embodiment of the novel method for enhancing epidermal stem cell growth in a subject comprises administering to a subject a topical composition comprising a therapeutic dosage of green algae and cyanobacteria. In one embodiment, the therapeutic administration of the novel composition of this invention induces the increase of epidermal pluripotent cells by as much as 25%, by as much as 50%, by as much as 100%, by as much as 150%, by as much as 200%, by as much as 300%, by as much as 400%, and by as much as 500%.

One embodiment of a method for enhancing a rate of nail growth in a subject comprises administering to a subject a topical composition according to this invention. In one embodiment, the therapeutic administration of the novel composition of this invention induces an increase in the rate of nail growth by as much as 200%.

One embodiment of a method for enhancing a rate of nail growth in a subject comprises administering to a subject a topical composition according to this invention and an enteral composition comprising a therapeutic dosage of green algae and a therapeutic dosage of cyanobacteria. In one embodiment, the therapeutic administration of the novel composition of this invention induces an increase in the rate of nail growth by as much as 200%.

One embodiment of a method for increasing a rate of proliferation of epidermal cells in a subject comprises administering to a subject a topical composition according to this invention. In one embodiment, the therapeutic administration of the novel composition of this invention induces an increase in the rate of epidermal cell proliferation by as much as 200%.

One method for increasing a rate of proliferation of epidermal stem cells in a subject comprises administering to a subject a topical composition according to this invention and administration of an enteral composition comprising a therapeutic dosage of green algae and a therapeutic dosage of cyanobacteria.

One embodiment of a method for enhancing a rate of hair growth in a subject comprises administering to a subject a topical composition according to this invention. In one embodiment, the therapeutic administration of the novel composition of this invention induces an increase in the rate of hair growth by as much as 200%. In particular, hair growth enhancement is beneficial following conventional transplant, cloning or injection of epidermal stem cells in the hair follicle region.

One embodiment of a method for enhancing a rate of hair growth in a subject comprises administering to a subject a topical composition according to this invention and an enteral composition comprising a therapeutic dosage of green algae and a therapeutic dosage of cyanobacteria. In one embodiment, the therapeutic administration of the novel composition of this invention induces an increase in the rate of hair growth by as much as 200%.

### Novel Enteral Composition and Method

One method for enhancing the production of pluripotent cells and increasing the longevity of said pluripotent cells in a subject comprises administering to a human an enteral composition comprising a therapeutic dosage comprising green algae and cyanobacteria, thereby enhancing pluripotent cell concentration in a subject. In one embodiment, the antioxidant activity in the novel composition induces the longevity of pluripotent cells, and thus an increase of circulating transient pluripotent cells and the concentration at enhanced concentration sites, by more than about 4 hours, by more than about 6 hours, by more than about 8 hours, by more than about 12 hours, by more than about 24 hours, by more than about 36 hours, by more than about 48 hours, and by more than about 72 hours. This can dramatically increase the effectiveness of the pluripotent cells in treating disease both at normal levels and the increased levels during the transient period.

In one embodiment, a composition for enhancing health and disease control in humans comprises a therapeutic dosage comprising green algae and cyanobacteria. In one embodiment, said green algae comprises a green algae selected from the group consisting of Haemotococcus pluvialis, Chlorella vulgaris, Chlorella pyrenoidosa, Dunaliella salina or a combination thereof. In one embodiment, said cyanobacteria comprises a cyanobacteria selected from the group consisting of Aphanizomenon flos-aquae, Spirulina pacifica, Spirulina plantesis or a combination thereof. In a preferred embodiment, said composition comprises a form selected from the group consisting of a dry powder, frozen liquid, liquid suspension and tablet. The novel algae composition provides therapeutic and prophylactic health benefits in the nature of protection of cellular tissues from the degenerative processes of free radicals and aging.

In one embodiment, a novel algae composition further comprises inert ingredients such as pharmaceutically acceptable carriers, texture enhancers and palatability enhancers. In a preferred embodiment, said palatability enhancer comprises xylitol.

This potential therapy option for various pathologies focuses on the body's own self healing mechanisms rather than invasive surgeries or pharmacological treatment regimens with potential adverse side effects and drug interactions. The aging population, the increasing demand for active retirement lifestyles and quality of life, and the ever increasing costs of medical care provide a climate for serious consideration of these alternative and holistic alternatives.

The health benefits of cyanobacteria and green algae, as well as their antioxidants, are acknowledged through historical usage. However, the use of each independently has limited value to the recipient. The increased number of pluripotent cells would be of little value if they were quickly destroyed by oxidants. Similarly, the use of antioxidants would be of marginal value where there existed only an insignificant number of living pluripotent cells due to aging processes. The combination of these natural products, the cyanobacteria and green algae, has a synergetic effect. Chemicals and/or components in the cyanobacteria assist in increasing the concentration of and/or extending the lifetime of the pluripotent cells. At the same time, the antioxidants in the green algae assist in the protection of these cells from the ravaging effects of oxidation and/or extending the pluripotent cell lifetime.

In one embodiment, it is the increased dosage and unique combination of algae in the novel composition, as compared to other commercially available supplements, that induces these results.

While a dietary supplement may be used by anyone who wishes to enhance their pluripotent cell production/lifetime/homing/activity, physical stamina and endurance in motor function, or general health, the algae supplement is particularly beneficial for elderly people, infirmed people and athletes where natural tissue regeneration functions may be depressed or improved tissue regeneration capabilities are necessary. Each of the constituent algae and microbial components has an individual tendency to enhance physical well being. However, the combination of cyanobacteria (e.g., AFA) and a green algae (e.g., H. pluvialis) (and optionally with other algae extracts and natural supporting components), when administered in proper concentration, stimulates pluripotent cell production/lifetime/homing/activity and physical stamina and endurance in motor function. The synergism between all of the components render the administration of a combination composition containing each algae and microbial component desirable.

In one embodiment, pluripotent cell concentration is increased at the enhanced concentration site. Such enhanced concentrations are the circulatory system, the skin, the nails, the hair and hair follicles, a diseased site or an injured site.

In a preferred embodiment, a novel algae composition further comprises a second species of cyanobacteria and a second species of green algae. The primary active components are antioxidants. The inclusion of additional antioxidant components permits the user to selectively optimize the ratio of algae to antioxidant to obtain a desired effect or synergism for a particular treatment regimen.

Applicant believes that whole foods provide the optimum nutritional sources. As opposed to purified or isolated vitamins and minerals, or concentrated vitamin compositions, whole foods contain supporting elements that synergistically enhance the individual activity of each component—that is, the micro-ecosystem has the optimum balance of ambient proteins, fats, saccharides, vitamins, minerals and the like to achieve the highest activity levels of each individual component. Thus, whole plant components and whole plant extracts are preferably used in the manufacture of the novel algae composition of this invention. Although less preferable, synthesized components and fractional components of the whole algae may also be used.

In one embodiment, the cyanobacteria and/or green algae provide enhanced concentration of certain trace minerals.

Different compositions of the invention are contemplated, as well as different methods of delivery and administration to the patient.

By way of illustration, but not limitation, an enteral composition may be formed into a pill (e.g., tablet), dry powder, liquid suspension or oral spray according to the teachings of U.S. Pat. Nos. 6,586,018 (algae composition), 7,081,259 (algae, extract having therapeutic activity on injuries, and pharmaceutical composition and health food containing the same), 6,852,344 (algae composition for treating CD34+ acute and chronic myeloid leukemia and a method thereof, and the like. The entire disclosure of each of said patents is hereby incorporated by reference into this specification. The novel composition may also be formed into a freeze dried product, frozen liquid, oleoresin or supercritical fluid extraction. Additionally, as will be described in more detail below, a powder or liquid nutritional supplement mixture may be formed for prepackaged distribution and mixed directly with food products by the user.

### Novel Topical Composition and Method

The topical compositions according to the invention are preferably dermatological compositions applied topically to the skin. The enteral composition according to this invention can be formed into a liquid suspension or paste and applied topically to the skin without any additional ingredients.

Additionally, as will be described in more detail below, the cosmetic compositions according to the invention can also be made available in any form known in the field of cosmetology including creams, emulsions, milks, sprays, solutions (both aqueous and hydro-alcoholic), anhydrous bases (such as lipsticks and powders), gels, ointments, moisturizing cream, skin benefit creams and lotions, foundation, night cream, lipstick, cleansers, toners, masks, color cosmetic products or by other method or any combination of the forgoing as would be known to one of ordinary skill in the art. The composition is most preferably used in anti-aging products for the face and other body parts, most especially leave-on products.

Additionally, the topical compositions according to the invention can also be made available in the form of hair and scalp products such as shampoos, conditioners, treatments, leave in hair products, mouse, hairspray, gel and other styling aids.

### Pluripotent Cells and Regenerative Processes in Living Organisms

Pluripotent cells play a role in the continuous lifelong physiological replenishment, healing and regenerative processes of various tissues and organs in living organisms. Pluripotent cells are capable of differentiating into more specialized cells such as myocytes, hepatocytes, osteocytes, glial cells, neurons, fibroblast or fibroblast-like cells, liver cells, myocardium cells, epithelial cells, brain cells, organ cells, and the like. Hematopoietic stem cells can also differentiate into many different types of blood cells including red blood cells, platelets and leukocytes. Hematopoietic stem cells are quite abundant and play a role in the continuous lifelong replenishment of blood cells.

Therefore, activation and enhancement of pluripotent cell production can provide regenerative mechanisms for tissue cells that are at the end of their life cycle, damaged or stressed. The beneficial effects are further optimized by enhanced homing of the stem cells and simultaneous stimulation of supporting systems such as the circulatory system, respiratory system, nervous system, gastrointestinal system, and the like.

The expeditious replacement of damaged tissue is critical to the return of the organ or tissue to its normal function and the prevention and/or reduction of the colonization of unwanted pathogens. The availability of on-site pluripotent cells (e.g., stem, stromal or basal cells) are a mandatory part of the replacement process. The rate of replacement is dependent upon many factors. These may include, but are not limited to, the ability and/or signaling process to induce the pluripotent adult stem cell (i.e. basal or stromal) to produce “daughter” cells, the condition of the circulatory system to move the pluripotent cells and/or nutrients to the appropriate site, and the availability of active pluripotent cells.

Pluripotent cell lifetime is an important aspect of the replacement rate of tissue. Many stem cells do not contain the ability to clone themselves. Thus, when they die or become inactive, reproductive function at that site is limited or fails completely. One demonstrative example might be baldness. As basal cells in the hair follicles die, hair regeneration ceases and one becomes bald. In the case of non-existent pluripotent cells, only transplantation would be an effective treatment.

Much research has been accomplished and is on-going to better understand factors that determine the lifetime of various pluripotent cells. While the exact nature of the aging process is not well understood, it has been determined that the lifetime of certain pluripotent cells (e.g. stem cells) may be extended by chemical treatment. Research has demonstrated the use of natural products such as cyanobacteria and green algae may be helpful in discrete cases as they contain many beneficial chemicals. Some research suggests that beneficial chemicals in cyanobacteria and green algae may include astaxanthin, L-selectin ligands, potent antioxidants, and the like. Potent antioxidants may include, for example, astaxanthin, zeaxthanin, 9-cis-beta-carotene and beta-CDS. The algae stimulates cytokines and interleukins.

Another factor for determining the lifetime of pluripotent cells is the susceptibility and/or rate of attack by detrimental chemical toxins. These may be in the form antibodies, enzymes, or just “toxic” chemicals. The oxidation process may either denature specific plasma proteins and restrict the osmosis of essential nutrients into the cell or interfere with the metabolic cell processes. In either case, eventually the cell ceases to function. Sources of these oxidants (free radical generators) include, but are not limited to, ingestion and/or metabolism byproducts of the host or parasitic microorganisms. These oxidants are often specific to the cell type or cytoplasm they attack. The use of antioxidants to reduce the destruction of cells has been well demonstrated in scientific literature.

The use of antioxidants (free radical scavengers) can extend the useful lifetime of pluripotent cells. These antioxidants can either be free floating in the circulatory or lymphatic system or, more commonly, attached to a cell wall. These antioxidants chemically react with free radical oxidants, neutralizing the oxidant to obviate any oxidation damage to the cell.

Research with diabetic patients revealed that the non-healing in epithelial necrosis of severe diabetics was caused due to “poor circulation” or the inability to get nutrients and disease fighting drugs to the site. More recent studies have observed the lack of stem cells at the site, suggesting this may also be a contributing factor. Clinical studies have shown that the presence of high quantities of cell damaging oxidants destroy pluripotent cells. There is some evidence that topical treatments containing high doses of antioxidants and steroids accelerate the healing of such wounds. The steroids (growth hormones) are thought to be pluripotent cell initiators in this case.

Adult stem cells are found most abundantly in bone marrow. The majority of the stem cells are slowly cycling such that less than about 1% of bone marrow cells are stem cells and of those, only about 10% are cycling at a given time. Stem cell production decreases with age. It is believed that stem cell production decreases in humans by 0.5% with each year beyond age 20.

Increasing pluripotent cell production, especially in elderly persons, is desirable for optimum health. Regeneration of deteriorating tissues prevents malfunctioning and disease. It is equally important to increase the activity of and prolong the life cycle of the pluripotent cells for the regenerative benefits to be realized. Thus, it is desirable to provide a compound that enhances pluripotent cell production, enhances its emission into the bloodstream, increases the activity of the pluripotent cells, prolongs the life cycle of the pluripotent cells, and optimizes the general physiological condition of the subject so the beneficial regenerative effects can be realized by absorption in said stressed tissues.

Disease management is paramount to the health and well being of living organisms. A normal function of the auto immune system is to routinely remove pathogens as they enter our bodies through the various cavities (e.g., mouth, nose, eyes) or penetrate our epithelial system (skin). A second function of our immune system is to attack colonies of pathogens that form in our tissue (e.g., abscesses). Both of these functions are generally performed by the white blood cells in our circulatory system.

There are three general types of white blood cells in our system. Lymphocytes constitute approximately 15-20% of the white blood cells. These lymphocytes contain antibodies, enzymes, and other chemicals used in the destruction of viruses. Monocytes (3-8%) carry antibodies to treat specific diseases. Granulocytes constitute about 75% of the white blood cells in our blood. These are cells that contain nonspecific antibodies, enzymes, and other chemicals for fighting bacterial infections.

The general concentration of the white blood cells is about 8,000 cells per cubic millimeter, but can increase to tens of thousands during an active infection in our body. These cells are produced by the stromal stem cells in the bone marrow. Although an excess of these white blood cells are stored in the bone marrow and other locations, during an active infection, it is necessary for the stromal cells to rapidly reproduce new white blood cells. The rate white blood cells are reproduced is dependent upon the quantity of available stromal cells in the bone marrow.

Granulocytes may be monitored via simple microscopy to identify the life stage of each cell. The stromal cell “daughters,” often referred to as “blast” cells, are very seldom observed in the blood sample since they are generally concentrated in the bone marrow. In blast cells, the nucleus appears as round red ball consuming approximately 90% of the cell when stained with Wright's stain. The nucleus of the next stage, “juvenile cells”, appears as a long curved band-like structure in the cell. The nucleus of “mature cells” appear as three separate segments. The nucleus of final stage and least active “hypersegmented cells” appear as 5-10 segments.

Thus, during an infection where there is an increased need for the granulocytes in the blood system, it is easy to identify the source of the cells. If the cells are coming from a stored source, the amount of mature cells (nucleus appearing as segments) relative to the number of immature cells (nucleus appearing as bands) will increase as compared to the normal ratio. If someone has taken a stimulant to increase the number and/or activity of the stromal cells, the increased production of granulocytes may be accounted for by the higher ratio of immature cells (nucleus appearing as bands) in the blood.

### Components of the Algae Composition

Most antioxidants are obtained by the ingestion of natural products such as fruits and vegetables. An alternative source is from non-pathogenic microbacteria and algae such as AFA, H. pluvialis, Spirulina pacifica, Spirulina plantesis, Dunaliella salina, Chlorella vulgaris and Chlorella pyrenoidosa. They produce antioxidants which appear to be more specific for the reduced destruction of pluripotent cells by oxidants generated from either decomposing tissue, pathogenic bacteria, aging processes and/or normal metabolic processes. The synergism between the cyanobacteria and green algae is an important aspect of this novel algae composition.

The synergism is, in part, due to the homologous molecular phylogenetic tree. The fewest mutations between species is preferred when selecting algae to combine together. Employing bioinformatics modeling, the “Deltas” assigned to correlate the nearness of various organic genus are used to determine the homological nearness of said genus and specie. These differences or “Deltas” can be grouped into separate families that provide evolutionary nearness measures that can be used to group family organisms into superior compositions that have powerful positive interactions. The evolutionary impetus has been to maximize the survival of the species.

The ingredients for a typical novel algae composition according to this invention are set out in proportions of the individual cyanobacteria and algae and may be varied to optimize the formulation to suit the specific needs of the user. It is an important feature of the present invention that it contains a mixture and is not based on a single cyanobacteria or algae. An unexpected synergistic effect has been experienced by the inventor. The combination of a powerful antioxidant with the stem cells provided by AFA, the only known food to increase the body's natural production of, and thus the concentration of, stem cells, are synergistic in combination since the protective aspect of the antioxidants serve to prolong the life cycle of the pluripotent cells, enhance the activity level of the pluripotent cells, and decrease the demand for tissue regeneration.

In one embodiment, about 2 to about 10 grams of cyanobacteria is administrated per day to a subject. In one embodiment, about 1 to about 10 grams of green algae is administered per day to a subject. In one embodiment, the green algae comprises from about 0.01 to about 50 weight percent of antioxidants, preferably from about 0.01 to about 10.0 weight percent, more preferably from about 0.01 to about 1.0 weight percent, and most preferably from about 0.01 to about 0.05 weight percent. Those antioxidants preserve the pluripotent cells.

Improvements that have been subjectively experienced by the inventor applicant include an improved sense of well being, an improvement in appetite, an increase in endurance and strength during physical exertion, and an increase in vigor and enthusiasm in daily activities. The inventor has observed similar results when the novel composition is administered to his cat and hamsters.

Upon additional topical administration of the novel composition, improvements that have been subjectively experienced by the inventor applicant include an improved skin growth, enhanced skin self-repair, reduction of dandruff and generally improved skin appearance.

In a preferred embodiment, the novel algae composition of this invention comprises a portion of AFA of from about 2 grams to about 10 grams, preferably about 5 grams.

In a preferred embodiment, the novel algae composition of this invention comprises a portion of Spirulina of from about 2 grams to about 10 grams, preferably about 4 to about 5 grams.

In a preferred embodiment, the novel algae composition of this invention comprises a portion of H. pluvialis comprising of from about 2 milligrams to about 10 milligrams of astaxanthin, preferably about 5 milligrams.

In a preferred embodiment, the novel algae composition of this invention comprises a portion of Chlorella vulgaris and/or Chlorella pyrenoidosa of from about 1 gram to about 4 grams, preferably about 2 grams. In a preferred embodiment, the novel algae composition of this invention comprises a portion of Chlorella vulgaris and/or Chlorella pyrenoidosa of from about 2 grams to about 10 grams, preferably about 4 to about 5 grams.

Spirulina contains antioxidants including antioxidant vitamins beta carotene (provitamin A) C, and E as well as antioxidant minerals selenium, manganese, zinc, copper, iron and chromium; phytonutrients such as phycocyanin, beta carotene, and polysaccharides; probiotics; nutraceuticals; gamma-linolenic acid (GLA); digestible protein; and sulfolipids. As a food, the algae contain all essential amino acids and many of the essential natural sugars. Its therapeutic action includes strengthening the immune system; supporting cardiovascular function and healthy cholesterol levels; improving gastrointestinal and digestive health; enhancing natural cleansing and detoxification; reducing cancer risks with antioxidant protection; inhibiting in vitro replication of HIV-1, Herpes, Influenza, Mumps and Measles; and enhancing stem cell growth. Synergistically, Spirulina works well with Chlorella.

In one embodiment, the micro algae Dunaliella salina is substituted for the H. Pluvialis. In one embodiment, the micro algae Dunaliella salina is added to the H. Pluvialis. Dunaliella salina provides a high source of astaxanathin, beta-CDS, 9-cis-beta-carotene and zeaxanthin.

Effective dosages of beta-CDS and 9-cis-beta-carotene in one embodiment of the novel composition and method of this invention are from about 10 mg to about 20 mg, preferably about 15 mg per day. Effective dosages of zeaxanthin in one embodiment of the novel composition and method of this invention are from about 500 mcg to about 1000 mcg, preferably about 800 mcg per day. Absorption is increased if the effective dosage is taken with fat sources.

In one embodiment, the novel composition and method of this invention comprises an immunoglobin such as beta-CDS. From about 10 mg to about 20 mg, preferably about 15 mg, of beta-CDS are in one embodiment.

In one embodiment, the novel composition of this invention, and the method of using the same, comprises green algae comprising from about 0.01 to about 50.0 weight percent of micropolysaccharides that bind with environmental toxins.

In one embodiment, the novel composition of this invention, and the method of using the same, comprises green algae comprising an antioxidant selected from the group consisting of zeaxanthin, astaxanthin, 9-CIS-beta carotene and beta-CDS.

Dunaliella comprises 9-cis-beta-carotene, a particularly potent antioxidant that has been shown to be as much as ten times more potent than other beta carotenes at preventing cancers. Dunaliella also comprises beta-CDS, a beta-carotene that has been shown to be an effective anti-cancer agent. Dunaliella also comprises zeaxanthin, a carotenoids that has been shown to be an effective treatment for age related macular degeneration. Dunaliella salina increases the activity of the immune system's macrophages and spleen cells.

There are several species of Chlorella. Those most commonly used in nutritional supplements are Chlorella vulgaris and Chlorella pyrenoidosa. Since Chlorella has a true nucleus, it is a later evolved organism than the cyanobacteria and is symbiotic with its primordial ancestor of a similar evolutionary time. Chlorella is much higher in chlorophyll content than other consumable algae varieties (often ranging from 3 to 5% pure natural chlorophyll). Chlorella's tough cell walls are made of complex polysaccharides, which have been shown to stimulate interferon production, exhibit strong anti-tumor activity, and provide super detoxifying properties. Chlorella contains more chlorophyll per gram than any other plant. Chlorella contains protein, carbohydrates, all of the B vitamins, vitamins C and E, amino acids, beta-carotene, rare trace minerals, Chlorella Growth Factor (CGF), chlorophyll, carotenoids, such as astaxanthin, canthaxanthin, flavoxanthin, loraxanthin, neoxanthin, violaxanthin, and echinenone. Therapeutic action of Chlorella includes neutralizing and/or eliminating toxins, pesticides and heavy metals from the body such as DDT, PCB, mercury, cadmium and lead; strengthening the immune system response; rebuilding nerve damage in the brain and nervous system; improving digestion; accelerating healing; protecting against radiation; aiding in the prevention of degenerative diseases; helping in treatment of Candida albicans; relieving arthritis pain; and because of its nutritional content, aiding in the success of numerous weight loss programs. Synergistically, Chlorella works well with Spirulina.

Chlorella, a green algae, contains greater levels of growth factors and clorellan are also present, as well as unique polysaccharide and cell wall compounds. The additional carotenoids in Chlorella work synergistically with beta carotene in cyanobacteria, providing enhanced anti-oxidant protection.

Chlorella has a unique cell chemistry that imparts immune-enhancing properties via the mucopolysaccharides' ability to bind with environmental toxins and carry them safely out of the body. The Chlorella cell wall's complex polysaccharides have been shown to stimulate interferon production and exhibit strong anti-tumor activity.

Chlorella's general rebuilding and detoxifying properties enhance the benefits of the stem cell regenerative processes. Chlorella builds vitality, improves digestion, enhances energy, improves mental clarity, and generally aids with other similar “lack of vitality” disorders.

Similarly, Chlorella's general immune system enhancing properties enhance the benefits of the stem cell regenerative processes. Chlorella stimulates the growth of friendly bacteria, which in turn has the probiotic effect of strengthening gut flora and resisting disease. Chlorella helps protect the body in its fight against both viruses and cancer. It increases macrophage activity and exhibits DNA repair mechanisms. Chlorella increases red blood cells, white blood cells, platelets, and albumin (many people with cancer have a decreased level of albumin). Treatment of viruses and fungi which sap energy, such as candida-overgrowth, Epstein-Barr virus, chronic fatigue immune deficiency syndrome (CFIDS), and AIDS, is advanced by the immune-enhancing qualities of CGF and antiviral effect of chlorophyll.

Chlorella may provide saccharides such as Chlorella polysaccharide N.β-1.3 glucan and monosaccharides glucuronic acid, arabinose, glucose, and fructose a- and β-anomers. These saccharides work synergistically with the cyanobacteria to enhance homing and cell signaling properties.

Chlorella's Growth Factor helps repair nerve tissues. The GLA content of Spirulina combined with the CGF content of Chlorella act together synergistically: GLA feeds the brain and nervous system with the nutrients while the CGF repairs damaged nerve tissue. This nerve repairing property complements and supports the stem cell regeneration processes.

The nucleic acid found in Chlorella (RNA/DNA) is a descendant of the cyanobacteria and provides a symbiosis (related to their origin in evolutionary time) for directing cellular renewal, growth, and repair. The amount of nucleic acid in the body decreases with age, and insufficient nucleic acid causes premature aging and weakened immunity. This DNA repairing property prevents disease and deterioration of tissues, complementing and supporting the stem cell regeneration processes.

Chlorella contains a greater quantity of fatty acids than Spirulina with approximately 20% constituting the artery-cleansing, omega-3, alpha-linolenic variety. Enhancing circulatory system health promotes the transport of the stem cells to the tissues where they are needed, supporting and enhancing the cyanobacteria's stem cell regeneration processes.

Similarly, well functioning body systems will be more receptive to, and benefit more from, the regeneration processes offered by the additional pluripotent cell production. By combining additional cyanobacteria (e.g., Spirulina) and/or green algae (e.g., Chlorella) components that enhance the general health of the subject and inhibit the harmful effects of disease causing organisms, the regenerative process benefits will be amplified.

In one embodiment, additional cyanobacteria and/or algae components are added for more synergistic effects. Optionally, the cyanobacteria-algae mixture may contain other algae, micro bacterial, herbal or cosmetic constituents as desired by the user and/or manufacturer. The user may selectively add algae and/or other components that are directed to provide alimentary or skin growth enhancing benefits desirable for the user. It is important, however, that the additional components selected do not impair the functionality of the cyanobacteria or green algae. Illustrative examples of additional components are set forth in greater detail below.

One commercially available source of cyanobacteria is Now Foods, Bloomingdale, Ill. 60108. One commercially available source of xylitol is Xylosweet™ available through Xlear, Inc., P.O. Box 970911, Orem, Utah 84097 www.xlearinc.com. One commercially available source of Chlorella is “nanized Chlorella concentrate” by Premier Research Labs, Round Rock, Tex. 78664. One commercially available source of xanthan gum is NOW FOODS, Bloomingdale, Ill. 60108 www.nowfoods.com. One commercially available source of astaxanthin is Mera Pharmaceutics, Kailua-Kona, Hi. 96740. One commercially available source of seaweed extract is DHC Corporation, 2-7-1 Minami-azabu, Minato-Ku, Tokyo. One commercially available source of live AFA in frozen form is (sold under the trade name “E3 Live”) is Vision at 310 Broad Street, P.O. Box N, Klamath Falls, Oreg. 97601.

### Administration of the Novel Enteral Composition

In one embodiment, a novel algae composition comprising a cyanobacteria, such as Aphanizomenon flos aquae (AFA), and a green algae, such as Haematococcus Pluvialis (H. Pluvialis), is administered to a subject. In some embodiments, the subject consumes and digests whole cells or extracts of said cyanobacteria and green algae. The cells may be fresh, frozen, liquid frozen, freeze-dried, dehydrated, or preserved in some other manner. Preferably, the whole cell extracts are in powdered or frozen liquid form.

This effective amount may be administered at a given frequency, such as about once a week, about twice a week, about three times a week, once a day, about twice a day, about three times a day, and the like. Preferably, the effective amount is administered two to four times per day, at least 4 hours apart, over a prolonged period of time, e.g. months or years.

The dietary supplement is preferably administered orally. This may in the form or tablets, liquid, powder, freeze dried product, or liquid suspension. Active ingredients comprise Aphanizomenon flos-aquae and Haemotococcus pluvialis. Optionally, active ingredients may also include Spirulina pacifica and/or Chlorella vulgaris and Chlorella pyrenoidosa.

The effective amount of the novel algae composition of this invention and frequency of administration may depend on a variety of factors, such as the algae components utilized, the general health of the subject being treated, and the physiological characteristics (e.g., height, age, weight, body fat percentage, metabolism, etc.) of the subject being treated.

For illustrative purposes, ranges of said active ingredients are shown below in weight percentage of the total weight of said active ingredients:

| | |
|---|---|
| Aphanizomenon flos-aquae | 30-70% |
| Haematococcus pluvialis or Dunaliella salina | 30-70% |
| Spirulina pacifica or Spirulina plantesis | 20-30% |
| Chlorella vulgaris or Chlorella pyrenoidosa | 10-20% |

Oral dosage may be administered in a single dosage or multiple dosages over the course of a day. Daily dosage of said active ingredients is to be from about 12 to about 20 grams daily.

It would be difficult to “overdose” on the novel composition because the subject would likely become full and lose any appetite for further ingestion. In one embodiment, it is preferred that Spirulina administered for disease fighting therapy is about 20 grams daily. In one embodiment, it is preferred that Spirulina administered for prophylactic therapy is about 10 grams daily. In one embodiment, it is preferred that Chlorella administered for disease fighting therapy is about 20 grams daily. In one embodiment, it is preferred that Chlorella administered for prophylactic therapy is about 5 grams daily.

Inert ingredients may optionally be added to the novel composition. Depending upon the form the supplement is to be administered, the mixture may contain inert ingredients including, but not limited to, pharmaceutically acceptable carriers, texture enhancers, palatability enhancers, flavorings, sweeteners, surfactants, solid bulk diluents, binders, and preservatives.

Pharmaceutically acceptable carriers function to assist in the administration of the therapeutic moieties. These are well known in the art and include compounds such as water, lipids, salts, surfactants, buffers, thickeners, preservatives, and the like.

One of the advantageous features of the novel compound is its flexibility and versatility for oral consumption by humans. Taste and texture are important characteristics of foods eaten by humans. Different people may have different preferences. The novel compound of this invention may be selectively adjusted by a user to meet the unique user's desirable qualities. By way of example, but not limitation, a user may adjust the amount of texture enhancer (e.g. xanthan gum, guar gum and/or combination thereof) and/or taste enhancer (e.g. xylitol) to produce a compound of desirable sweetness and texture. By way of illustration, adding more texture and/or taste enhancer (taking care not to exceed the maximum limits) produces a compound that may be used in the form of a milkshake, pudding, or ice cream topping.

Xylitol is a sweetener that promotes dental health and oral health, has antibacterial properties, and does not produce the harmful health effects of processed and refined sugars. Xylitol has a Glycemic Index of 7, making it particularly well suited for diabetics.

By way of further example, the novel compound may be mixed with soy milk, gelatin, milk powder, milk, berries, chocolate syrup, or juice to increase the variety of oral administration options. This is especially useful for elderly or ill subjects who have depressed appetites.

In one embodiment, the novel algae composition is formed into a powder. Such powder is prepackaged in individual “packet” doses or in a larger bulk container where the user measures spoonfuls or scoopfuls to mix with food products.

In one embodiment, food products containing the novel algae compound are prepared and prepackaged for sale. By way of illustration, but not limitation, such food products include shakes, juices, solid snack foods, candies, desserts, soups, hot drinks, and the like.

The following examples are provided to illustrate particular features of various described embodiments. The scope of the present invention should not be limited to those features exemplified.

In one preferred embodiment, the novel composition comprises active and inert ingredients in powdered form that may be formed into shakes, puddings and the like. In one embodiment, the ingredients are blended with a high speed blender such as a Vita-Mix 5000. This method of blending ensures that algae with relatively strong cellular membranes are made bio-available. In a preferred embodiment containing xylitol as a taste enhancer, the xylitol is added early in the mix sequence to provide a pH range that is friendly to the algae components.

### EXAMPLE 1

### Active Ingredients

| Algae Component | Weight |
|---|---|
| Aphanizomenon flos-aquae | 2 g-10 g |
| Astaxanthin from H. pluvialis or Dunaliella salina | 2 mg-10 mg |
| Spirulina plantesis or Spirulina pacifica | 2 g-9 g |
| Chlorella vulgaris or Chlorella pyrenoidosa | 1 g-4 g |

### EXAMPLE 2

### Active Ingredients

| Algae Component | Weight |
|---|---|
| Aphanizomenon flos-aquae (AFA) | 5 g |
| Astaxanthin from H. pluvialis or Dunaliella salina | 5 mg |
| Spirulina pacifica or Spirulina plantesis | 4 g |
| Chlorella vulgaris or Chlorella pyrenoidosa | 2 g |

### EXAMPLE 3

### Active Ingredients

| Algae Component | Weight |
|---|---|
| Aphanizomenon flos-aquae (AFA) | 5 g |
| Astaxanthin from H. pluvialis or Dunaliella salina | 5 mg |
| Spirulina pacifica or Spirulina placentas | 4 g |
| Chlorella vulgaris or Chlorella pyrenoidosa | 2 g |

The therapeutic dosage of astaxanthin may be obtained from about 1 gram to about 10 grams of H. pluvialis or Dunaliella salina. In one embodiment, the astaxanthin is derived from both H. pluvialis or Dunaliella salina.

### EXAMPLE 4

### Active Ingredients

| Algae Component | Weight |
|---|---|
| Aphanizomenon flos-aquae | 2 g-10 g |
| H. pluvialis | 1 g-10 g |
| Spirulina plantesis or Spirulina pacifica | 2 g-9 g |
| Chlorella vulgaris or Chlorella pyrenoidosa | 1 g-4 g |
| Dunaliella salina | 1 g-10 g |

In one embodiment, a texture enhancer, preferably xanthan gum or guar gum, is added. (Xanthan gum also slows the digestive process and enhances the absorption of the cyanobacteria and algae elements.) In another embodiment, a taste or sweetness enhancer, preferably xylitol, is added. In one embodiment, both a taste and texture enhancer are added.

In one embodiment, additional cyanobacteria and/or algae components are added for more synergistic effects. Optionally, the cyanobacteria-algae mixture may contain other algae, micro bacterial or herbal constituents as desired by the user and/or manufacturer. The user may selectively add algae and/or microbial components and/or herbs that are directed to provide health benefits desirable for the user. It is important, however, that the micro bacterial and/or algae components and/or herbs selected do not impair the functionality of the cyanobacteria or green algae.

In a preferred embodiment, the novel algae composition of this invention comprises a portion of xylose of from about 2 grams to about 36 grams, preferably about 9 grams.

In a preferred embodiment, the novel algae composition of this invention comprises a portion of xanthan and/or guar gum of from about 1 gram to about 10 grams, preferably about 4 grams.

### EXAMPLE 5

### Active and Inert Ingredients

| Algae Component | Weight |
|---|---|
| Aphanizomenon flos-aquae | 2 g-10 g |
| Haematococcus pluvialis | 2 mg-10 mg |
| Spirulina plantesis or Spirulina pacifica | 2 g-9 g |
| Chlorella vulgaris or Chlorella pyrenoidosa | 1 g-4 g |
| Xylose | 2 g-36 g |
| Xanthan or Guar Gum | 1 g-10 g |

### EXAMPLE 6

### Active and Inert Ingredients

| Algae Component | Weight |
|---|---|
| Aphanizomenon flos-aquae | 2 g-10 g |
| Astaxanthin from H. pluvialis or Dunaliella salina | 2 mg-10 mg |
| Spirulina plantesis or Spirulina pacifica | 2 g-9 g |
| Chlorella vulgaris or Chlorella pyrenoidosa | 1 g-4 g |
| Xylose | 2 g-36 g |
| Xanthan or Guar Gum | 1 g-10 g |

### EXAMPLE 7

### A Frozen Concentrate

In one preferred embodiment, a frozen concentrate is prepared. This frozen concentrate may be unthawed and used as a pancake topping, ice cream topping, spread for crackers or bread, and the like.

Four ounces of water are heated to 100 degrees Celsius. One to one and one-quarter tablespoons of agar powder are added with constant agitation until the powder is dissolved. 10 frozen blueberries are added, ensuring that the temperature of the solution does not decrease below 60 degrees Celsius. Remove the solution from the heat source or otherwise discontinue heat.

To this solution is added 1 level tablespoon of liquid (from frozen) E3Live brand Aphanizomenon flos-aquae, 1 rounded teaspoon of powdered Spirulina pacifica, ¼ level teaspoons (2 capsules) of Solgar brand H. pluvialis capsules (containing 4 mg of astaxanthin), 1 rounded teaspoon of powdered Chlorella vulgaris, 1 heaping teaspoon of crystalline xylitol, 1 rounded teaspoon of powdered lecithin, 2 rounded tablespoons of soy powder, and 4 ounces distilled water.

Cool the solution, preferably in a freezer, for about one hour until a gel-like consistency is formed. Add four ounces of water and agitate the solution at a high speed for 30-60 seconds. This may be done by a culinary blender.

The solution may be frozen for long-term storage.

Thus, this embodiment of the composition comprised:

| Component | measurement (ml) | volume percent |
|---|---|---|
| Aphanizomenon flos-aquae | 15-17 | 6.67-9.51 |
| H. pluvialis (containing | 0.75-1.75 | 0.33-10.94 |
| 4 mg of astaxanthin) | | |
| Spirulina pacifica | 6-8 | 2.67-4.48 |
| Chlorella vulgaris | 6-8 | 2.67-4.48 |
| Xylitol | 7-9 | 3.11-5.03 |
| Lecithin | 6-8 | 2.67-4.48 |
| soy powder | 36-50 | 16.92-26.21 |
| distilled water | 300-425 | 66.45-80.68 |
| blueberries | 10-28 | 1.79-6.60 |
| agar powder | 15-22 | 2.63-5.38 |

Thus, this embodiment of the active ingredients of the composition comprised:

| Component | measurement (ml) | volume percent |
|---|---|---|
| Aphanizomenon flos-aquae | 15-17 | 45.80-57.14 |
| H. pluvialis (containing 4 mg | 0.75-1.75 | 2.22-6.09 |
| of astaxanthin) | | |
| Spirulina pacifica | 6-8 | 18.32-26.89 |
| Chlorella vulgaris | 6-8 | 18.32-26.89 |

### Administration of the Novel Topical Composition

The cosmetic compositions according to the invention comprise from about 0.1 to about 15%, preferably from about 0.5 to about 10%, of product active ingredients containing the therapeutic amount of the novel algae composition comprising a cyanobacteria, such as Aphanizomenon flos aquae(AFA), and a green algae, such as Haematococcus Pluvialis (H. Pluvialis) or Duniella salina. The following examples are provided to illustrate particular features of various described embodiments. The scope of the present invention should not be limited to those features exemplified. For illustrative purposes, ranges of said active ingredients are shown below in weight percentage of the total weight of said active ingredients:

| | |
|---|---|
| Aphanizomenon flos-aquae | 30-70% |
| Haematococcus pluvialis or Dunaliella salina | 30-70% |

Optionally, active ingredients may also include Spirulina pacifica and/or Chlorella vulgaris and Chlorella pyrenoidosa. For illustrative purposes, ranges of said active ingredients are shown below in weight percentage of the total weight of said active ingredients:

| | |
|---|---|
| Aphanizomenon flos-aquae | 30-70% |
| Haematococcus pluvialis or Dunaliella salina | 30-70% |
| Spirulina pacifica or Spirulina plantesis | 20-30% |
| Chlorella vulgaris or Chlorella pyrenoidosa | 10-20% |

Using a volume comparison, one embodiment of the active ingredients of the composition comprise:

| | |
|---|---|
| Aphanizomenon flos-aquae | 40-60% |
| Haematococcus pluvialis or Dunaliella salina | 0.5-5% |
| Spirulina pacifica or Spirulina plantesis | 10-40% |
| Chlorella vulgaris or Chlorella pyrenoidosa | 10-40% |

One preferred embodiment comprises the following active ingredients:

| Component | volume percent |
|---|---|
| Aphanizomenon flos-aquae | 45.80-57.14 |
| H. pluvialis (containing 4 mg of astaxanthin) | 2.22-6.09 |
| Spirulina pacifica | 18.32-26.89 |
| Chlorella vulgaris | 18.32-26.89 |

In one embodiment, the composition comprises an emulsion, cream, lotion, solution, anhydrous base, gel, ointment, or combination thereof. The active algae ingredients, e.g. the therapeutic dosage, the green algae and the cyanobacteria, contain antioxidants. In one embodiment, the green algae comprises from about 0.01 to about 10.0 weight percent of antioxidants. In one embodiment, the active ingredients comprise from about 0.01 to about 6.0 volume percent of astaxanthin.

In some embodiments, the cosmetic preparation comprises whole cells or extracts of said cyanobacteria and green algae. The cells may be fresh, frozen, freeze-dried, dehydrated, or preserved in some other manner. Preferably, the whole cell extracts are in powdered form or liquid frozen form.

This effective amount of the algae composition may be administered as a topical or cosmetic preparation at a given frequency, such as about once a week, about twice a week, about three times a week, once a day, about twice a day, about three times a day, and the like. Preferably, the effective amount is administered two to four times per day, at least 4 hours apart, over a prolonged period of time, e.g. months or years.

The effective amount of the novel algae composition of this invention and frequency of administration may depend on a variety of factors, such as the algae components utilized, the general health of the subject being treated, and the physiological and dermatological characteristics of the subject being treated.

Aging tissues experience greater stress and thus will benefit from the protective components of the algae composition. They will also benefit from the enhanced regenerative properties, e.g. stem cell enhancement, of the algae composition.

The rate of hair growth in a subject may be doubled by administering to a subject a topical composition according to this invention by contacting the scalp and hair follicles with the composition for at least 30 seconds. Preferably, the composition is in contact with the scalp and hair follicles for at least about 2 hours, at least about 4 hours, at least about 6 hours, at least about 8 hours, at least about 12 hours, at least about 24 hours, and at least about 36 hours.

This method has particular benefit when used with hair transplantation patients. Baldness is a consequence of no stem cell activity. Pattern baldness occurs most frequently in the frontal lobe area of the head. Hair transplants frequently relocate hair follicles from the top of the head to the frontal lobe area of the head. The newly transplanted hair follicles will benefit from the benefits of the enhanced stem cell proliferation and longevity. Satellite cells surrounding the transplanted hair follicles will also benefit from the stem cell growth. Enhanced hair growth will reduce the number of hair transplant surgeries that are required for a patient and improve the survivability of the transplanted hair follicles.

The rate of hair growth is further enhanced by administering to a subject a topical composition according to this invention and an enteral composition comprising a therapeutic dosage of green algae and a therapeutic dosage of cyanobacteria. In one embodiment, the therapeutic administration of the novel composition of this invention induces an increase in the rate of hair growth by as much as 200%.

The rate of nail growth in a subject may be doubled by administering to a subject a topical composition according to this invention by contacting the nails with the composition for at least 30 seconds. Preferably, the composition is in contact with the nail for at least about 2 hours, at least about 4 hours, at least about 6 hours, at least about 8 hours, at least about 12 hours, at least about 24 hours, and at least about 36 hours.

The rate of nail growth is further enhanced by administering to a subject a topical composition according to this invention and an enteral composition comprising a therapeutic dosage of green algae and a therapeutic dosage of cyanobacteria. In one embodiment, the therapeutic administration of the novel composition of this invention induces an increase in the rate of nail growth by as much as 200%.

The rate of skin growth in a subject may be doubled by administering to a subject a topical composition according to this invention by contacting the skin with the composition for at least 30 seconds. Preferably, the composition is in contact with the skin for at least about 2 hours, at least about 4 hours, at least about 6 hours, at least about 8 hours, at least about 12 hours, at least about 24 hours, and at least about 36 hours.

The rate of skin growth is further enhanced by administering to a subject a topical composition according to this invention and an enteral composition comprising a therapeutic dosage of green algae and a therapeutic dosage of cyanobacteria. In one embodiment, the therapeutic administration of the novel composition of this invention induces an increase in the rate of skin growth by as much as 200%.

The active ingredients may be mixed as a paste or a solution with water to form a simple topical composition according to this invention. This may be applied directly to the skin, scalp and fingernails. In other embodiments, more complex compositions may be formed with other components.

The preparation of cosmetic applications is well known in the art. Thus, by way of illustration, a cosmetic preparation may be prepared in accordance with the teachings of U.S. Pat. No. 7,025,966 (Compositions of marine botanicals to provide nutritional to aging and environmentally damaged skin) and/or U.S. Pat. No. 7,128,914 (Product containing an extract of red algae of the genus Porphyra and methods for protecting cells) as described below. The entire specification of each of these United States patents is incorporated by reference in its entirety for its teachings.

These compositions can also contain one or more formulation agents or additives of known and conventional use in cosmetic and dermatological compositions, including but not limited to softeners, colorants, film-forming agents, surface-active agents, perfumes, preservatives, emulsifiers, oils, glycols, sebum-absorption agents, vitamins and the like. Those skilled in the cosmetics art know which formulation agents to add to the compositions of the invention and in what quantities in relation to the desired properties. It is important, however, that the concentrations and combinations of the compounds and extracts be selected in such a way that the combinations are chemically compatible and do not form complexes which precipitate from the finished product. It is important that the additional components of the cosmetic preparation do not impair the functionality of the cyanobacteria or green algae active ingredients.

Compositions of the present invention can include other beneficial agents and compounds such as, for example, acute or chronic moisturizing agents (including, e.g., humectants, occlusive agents, and agents that affect the natural moisturization mechanisms of the skin), anti-oxidants, sunscreens having UVA and/or UVB protection, skin lightening agents (e.g. hydroquinone), emollients, anti-irritants, vitamins, trace metals, anti-microbial agents, botanical extracts, fragrances, and/or dyes and color ingredients.

Moisturizing agents that can be used with the compositions of the present invention include xylitol, amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturization factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, acetylated lanolin, acetylated lanolin alcohol, acrylates/C 10-30 alkyl acrylate crosspolymer, acrylates copolymer, alanine, algae extract, aloe barbadensis, aloe-barbadensis extract, aloe barbadensis gel, althea officinalis extract, aluminum starch octenylsuccinate, aluminum stearate, apricot (prunus armeniaca) kernel oil, arginine, arginine aspartate, arnica montana extract, ascorbic acid, ascorbyl palmitate, aspartic acid, avocado (persea gratissima) oil, barium sulfate, barrier sphingolipids, butyl alcohol, beeswax, behenyl alcohol, beta-sitosterol, BHT, birch (betula alba) bark extract, borage (borago officinalis) extract, 2-bromo-2-nitropropane-1,3-diol, butcherbroom (ruscus aculeatus) extract, butylene glycol, calendula officinalis extract, calendula officinalis oil, candelilla (euphorbia cerifera) wax, canola oil, caprylic/capric triglyceride, cardamon (elettaria cardamomum) oil, carnauba (copernicia cerifera) wax, carrageenan (chondrus crispus), carrot (daucus carota sativa) oil, castor (ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (anthemis nobilis) oil, cholesterol, cholesterol esters, cholesteryl hydroxystearate, citric acid, clary (salvia sclarea) oil, cocoa (theobroma cacao) butter, coco-caprylate/caprate, coconut (cocos nucifera) oil, collagen, collagen amino acids, corn (zea mays) oil, fatty acids, decyl oleate, dextrin, diazolidinyl urea, dimethicone copolyol, dimethiconol, dioctyl adipate, dioctyl succinate, dipentaerythrityl hexacaprylate/hexacaprate, DMDM hydantoin, DNA, erythritol, ethoxydiglycol, ethyl linoleate, eucalyptus globulus oil, evening primrose (oenothera biennis) oil, fatty acids, tructose, gelatin, geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (vitis vinifera) seed oil, hazel (corylus americana) nut oil, hazel (corylus avellana) nut oil, hexylene glycol, honey, hyaluronic acid, hybrid safflower (carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, imidazolidinyl urea, iodopropynyl butylcarbamate, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (asminum officinale) oil, jojoba (buxus chinensis) oil, kelp, kukui (aleurites moluccana) nut oil, lactamide MEA, laneth-16, laneth-10 acetate, lanolin, lanolin acid, lanolin alcohol, lanolin oil, lanolin wax, lavender (lavandula angustifolia) oil, lecithin, lemon (citrus medica limonum) oil, linoleic acid, linolenic acid, macadamia ternifolia nut oil, magnesium stearate, magnesium sulfate, maltitol, matricaria (chamomilla recutita) oil, methyl glucose sesquistearate, methylsilanol PCA, microcrystalline wax, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (olea europaea) oil, orange (citrus aurantium dulcis) oil, palm (elaeis guineensis) oil, palmitic acid, pantethine, panthenol, panthenyl ethyl ether, paraffin, PCA, peach (prunus persica) kernel oil, peanut (arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG40 stearate, PEG-50 stearate, PEG-100 stearate, PEG-150 stearate, pentadecalactone, peppermint (mentha piperita) oil, petrolatum, phospholipids, polyamino sugar condensate, polyglyceryl-3 diisostearate, polyquatemium-24, polysorbate 20, polysorbate 40, polysorbate 60, polysorbate 80, polysorbate 85, potassium myristate, potassium palmitate, potassium sorbate, potassium stearate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, quatemium-15, quatemium-18 hectorite, quaternium-22, retinol, retinyl palmitate, rice (oryza sativa) bran oil, RNA, rosemary (rosmarinus officinalis) oil, rose oil, safflower (carthamus tinctorius) oil, sage (salvia officinalis) oil, salicylic acid, sandalwood (santalum album) oil, serine, serum protein, sesame (sesamum indicum) oil, shea butter (butyrospermum parkii), silk powder, sodium chondroitin sulfate, sodium DNA, sodium hyaluronate, sodium lactate, sodium palmitate, sodium PCA, sodium polyglutamate, sodium stearate, soluble collagen, sorbic acid, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (triticum vulgare) germ oil, and ylang ylang (cananga odorata) oil.

The algae components provide a preferred source of antioxidants. Additional antioxidants that can additionally be used with the compositions of the present invention include acetyl cysteine, ascorbic acid, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl fuirfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

Protection against ultraviolet light is commonly a feature of cosmetic preparations. Compounds that have ultraviolet light absorbing properties that can be used with the compounds of the present invention include titanium dioxide, zinc oxide, benzophenone, benzophenone-1, benzophenone-2, benzophenone-3, benzophenone-4 benzophenone-5, benzophenone-6, benzophenone-7, benzophenone-8, benzophenone-9, benzophenone-10, benzophenone-11, benzophenone-12, benzyl salicylate, butyl PABA, cinnamate esters, cinoxate, DEA-methoxycinnamate, diisopropyl methyl cinnamate, ethyl dihydroxypropyl PABA, ethyl diisopropylcinnamate, ethyl methoxycinnamate, ethyl PABA, ethyl urocanate, glyceryl octanoate dimethoxycinnamate, glyceryl PABA, glycol salicylate, homosalate, isoamyl p-methoxycinnamate, PABA, PABA esters, Parsol 1789, and isopropylbenzyl salicylate.

Additional compounds and agents that can be used with the compositions of the present invention include skin lightening agents (e.g. kojic acid, hydroquinone, ascorbic acid and derivatives, retinoids and their derivatives, and niacinamide), emollients (e.g. esters and fatty acids), vitamins (e.g. D, E, A, K, and C), trace metals (e.g. zinc, calcium and selenium), anti-irritants (e.g. steroids and non-steroidal anti-inflammatories), antimicrobial agents (e.g. triclosan), botanical extracts (e.g. aloe vera, chamomile, cucumber extract, ginkgo bibloba, ginseng, and rosemary), dyes and color ingredients (e.g. D&C blue no. 4, D&C green no. 5, D&C orange no. 4, D&C red no. 17, D&C red no. 33, D&C violet no. 2, D&C yellow no. 10, D&C yellow no. 11 and DEA-cetyl phosphate), preservatives (e.g. BHA), emollients (i.e. organic esters, fatty acids, lanolin and its derivatives, plant and animal oils and fats, and di- and triglycerides), antimicrobial agents (e.g., triclosan and ethanol), and fragrances (natural and artificial).

The preparation of hair or scalp applications is well known in the art. These may include, but are not limited to, shampoos, conditioners, treatments, leave in hair products, mouse, hairspray, gel and other styling aids.

Detergents that can be used with the compositions of the present invention include surfactants, especially cocamidopropyl betaine and straight-chain alkyl benzene sulfonates such as Ammonium Lauryl Sulfate or ammonium lauryl ether sulfate.

Thickeners and emulsifiers that can be used with the compositions of the present invention include ammonium xylenesulfonate, Glycerol stearate, Sodium chloride (table salt), ammonium chloride and modified cellulose based thickeners.

Detergents enhancers that can be used with the compositions of the present invention include tetrasodium EDTA.

Foaming agents that can be used with the compositions of the present invention include Cocamide DEA (or MEA or TEA).

A foam stabilizing detergent that can be used with the compositions of the present invention include PEG-5 cocamide, a foam stabilizer, surfactant, and emulsifier.

A wax that can be used with the compositions of the present invention include glycol distearate.

Humectants that can be used with the compositions of the present invention include panthenol, Glycerine and propylene glycol (which is also a preservative).

Preservatives that can be used with the compositions of the present invention include DMDM hydantoin, imidazolidinyl urea, biocides, isothiazolinone, methylisothiazolinone, methylchloroisothiazolinone, Sodium benzoate, 2-bromo-2-nitropropane-1,3-diol.

Acidifiers that can be used with the compositions of the present invention include citric acid.

Buffers that can be used with the compositions of the present invention include sodium citrate.

Conditioning agents that can be used with the compositions of the present invention include Silicone oils such as dimethicone and cyclomethicone, long chain fatty alcohols like cetyl alcohol, oleyl alcohol and stearyl alcohol, cetrimonium chloride, quaternary ammonium compounds like stearalkonium chloride, disteardimonium chloride, quaternium-5 or quaternium-18, and polyquaternium-10 and emollients such as isopropyl palmitate.

Anti-dandruff agents that can be used with the compositions of the present invention include selenium sulfide, zinc pyrithione, salicylic acid and sulfur.

Colors and dyes that can be used with the compositions of the present invention include Yellow 5 (CI#19140), Urea, Red 33 (CI#17200), Blue 1 (CI#42090), Green 5, Ext. Violet 2, Green 8, Red 40, Yellow 6, FD+C Blue #2, D+C Red #27, D+C Yellow #10, and D+C Red #21.

Other additives that can be used with the compositions of the present invention include honey, various herb extracts, Aloe Barbadensis (Organic Aloe Vera) Leaf Juice, fragrances, silk or soy amino acids, other amino acids, surfactants, proteins, fragrance oils, essential oils, Nasturtium Officinate (Watercress) Extract, and Chamomilla Recutilla (Matricaria) Extract.

Other ingredients serving one or more of the aforementioned functions that can be used with the compositions of the present invention include Sodium C14-16, Olefin Sulfonate solution, Ammonium Lauryl Sulfate, Sodium Lauryl Sulfate, Cocamide DEA, Cocamidopropyl betaine, Sodium Laureth Sulfate, Cocamidopropyl Betaine, Cocamide DEA, PEG-150 Distearate, PEG-80, Sorbitan Laurate, Cocamidopropyl, Betaine, Sodium Trideceth Sulfate, Glycerin, Disodium Lauroamphodiacetate, Distearate & Laureth-13 Carboxylate, Sodium Laureth Sulfate, Disodium Laureth Sulfosuccinatemethylparaben, propylene glycol, Diazolidinyl Urea, phenoxyethanol (CAS # 122-99-6), methylparaben (CAS # 99-76-3), butylparaben (CAS # 94-26-8), ethylparaben (CAS # 12047-8), propylparaben (CAS # 94-13-3), Isopropylparaben, Isobutylparaben, Butylparaben, SODIUM HYDROXYMETHYLGLYCINATE, Sodium Laureth Sulfate, Glycol Stearate, D.I. Water, Cetyl Alcohol, Pyridinium Chloride, DL Panthenol, Vitamins A&E, Germaben II, Coconut Oil Acid Diethanolamine Condensate, Lauric Acid Diethanolamine Condensate, Oleic Acid Diethanolamine Condensate, Sodium Laureth Sulfate, Disodium Cocoamphodiacatate, Cocamidopropyl Betaine, Sodium Chloride, Tocopheryl Acetate (Vitamin E Acetate), Retinyl Palmitate (Vitamin A Palmitate), Panthenol (Pro-Vitamin B5), Magnesium Sulfate, Sodium Thiosulfate, DMDM Hydantoin, Iodopropynyl Butylcarbamate, Sodium Borate, Trisodium EDTA, Tetrasodium EDTA, and Disodium EDTA.

Applicant-inventor believes that pluripotent stem cells and/or components thereof, such as CD34, are disposed in the hair follicle region. Hair follicle stem cell growth may be enhanced in the inner sheath, outer sheath, sebaceous gland by the topical and/or enteral administration of the novel composition of this invention.

A method for increasing the longevity (i.e. lifetime) of hair follicle stem cells comprises administering a topical composition according to this invention one time per day. Administration is by contacting the hair follicles with the topical composition for at least 1 minute, for at least 3 minutes, for at least 5 minutes, for at least 15 minutes, for at least 30 minutes, for at least 60 minutes, for at least 4 hours, for at least 8 hours or for at least 12 hours. In one embodiment, the antioxidant activity in the novel composition of this invention induces the longevity of hair follicle stem cells by more than about 4 hours, by more than about 6 hours, by more than about 8 hours, by more than about 12 hours, by more than about 24 hours, by more than about 36 hours, by more than about 48 hours, and by more than about 72 hours. This can dramatically increase the effectiveness of the hair follicle stem cells in replicating and/or treating hair transplantation. In one embodiment of this method, the topical administration is combined with the enteral administration of the novel composition.

In accordance with this invention, there is also provided a method for increasing a rate of proliferation of hair follicle stem cells in a subject, comprising: administering to a subject a topical composition according to this invention. In one embodiment, the therapeutic administration of the novel composition of this invention induces an increase in the rate of hair follicle stem cell proliferation by as much as 200%.

### Experimental Results of Topical Administration

Inventor-applicant mixed 1 level tablespoon of liquid (from frozen) E3Live brand Aphanizomenon flos-aquae, 1 rounded teaspoon of powdered Spirulina pacifica, ¼ level teaspoon (2 capsules) of Solgar brand H. pluvialis capsules (containing 4 mg of astaxanthin), 1 rounded teaspoon of powdered Chlorella vulgaris, 1 heaping teaspoon of crystalline xylitol, 1 rounded teaspoon of powdered lecithin, 2 rounded tablespoons of soy powder, and 4 ounces distilled water. The composition was blended for 30 seconds on high with a Vita-Mix 5000 high speed blender. To avoid heat build up, blending was paused for 60 seconds. The composition was blended a second time for 30 seconds on high.

The balance of the mixture was orally consumed as a food supplement. This was also done once per day at the same time as the topical administration.

Within two minutes following blending, the composition was also topically applied to the fingernail region on the hands by contacting the fingers from the knuckles to the tips of the fingernails with the composition for at least 30 seconds and then allowed to dry at room temperature. During application, the fingernails of the right hand were immersed for at least 15 seconds longer than the fingernails of the left hand. It was applied once per day.

Thus, this embodiment of the composition comprised:

| Component | measurement (ml) | volume percent |
|---|---|---|
| Aphanizomenon flos-aquae | 15-17 | 6.67-9.51 |
| H. pluvialis (containing 4 mg | 0.75-1.75 | 0.33-10.94 |
| of astaxanthin) | | |
| Spirulina pacifica | 6-8 | 2.67-4.48 |
| Chlorella vulgaris | 6-8 | 2.67-4.48 |
| Xylitol | 7-9 | 3.11-5.03 |
| Lecithin | 6-8 | 2.67-4.48 |
| soy powder | 36-50 | 16.92-26.21 |
| distilled water | 100-125 | 49.57-61.96 |

Thus, this embodiment of the active ingredients of the composition comprised:

| Component | measurement (ml) | volume percent |
|---|---|---|
| Aphanizomenon flos-aquae | 15-17 | 45.80-57.14 |
| H. pluvialis (containing 4 mg | 0.75-1.75 | 2.22-6.09 |
| of astaxanthin) | | |
| Spirulina pacifica | 6-8 | 18.32-26.89 |
| Chlorella vulgaris | 6-8 | 18.32-26.89 |

This regimen was followed daily for a period of two months. After the two month period, the composition was administered as a food supplement and the topical administration ceased. The rate of nail growth slowed to the rate of growth of the applicant-inventor's fingernails with no or nominal topical administration of the composition.

Fingernails grow at an average rate of 0.01 centimeters per day, requiring 4-6 months to regrow completely. Actual growth rate is dependent upon age, health, season, exercise level and hereditary factors.

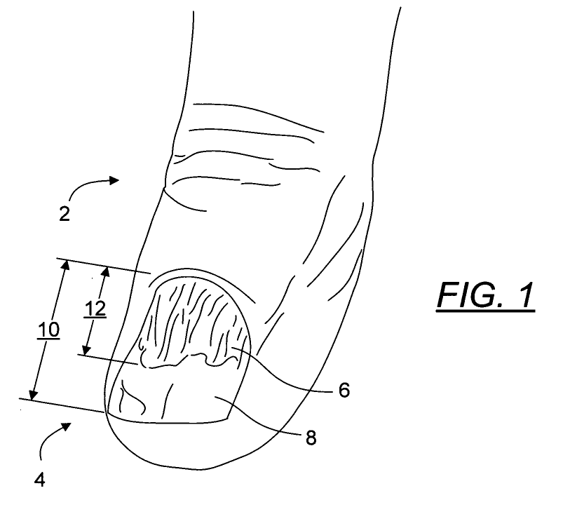

The Inventor-applicant observed that the nails where the composition was topically applied experienced a faster rate of growth by at least 200% as compared to the nail growth when the fingernails received little or no topical administration of the compound. Additionally, the texture of the new nail growth was visibly different from the old nail growth. FIG. 1 is a perspective view of a finger following treatment.

Referring to FIG. 1, a finger 2 with a fingernail 4 is depicted. The portion of new fingernail growth 6 has a length 12 of the total length 10 of fingernail 4. The portion of new fingernail growth 6 exhibited some vertical ridges that were not apparent in the portion of old nail 8. The portion of new fingernail growth 6 exhibited a different texture and color than the portion of old nail 8. The portion of new fingernail growth 6 appeared thinner and whiter in color than the portion of old nail 8 which was thicker, peeling, flaking and more yellowish in color.

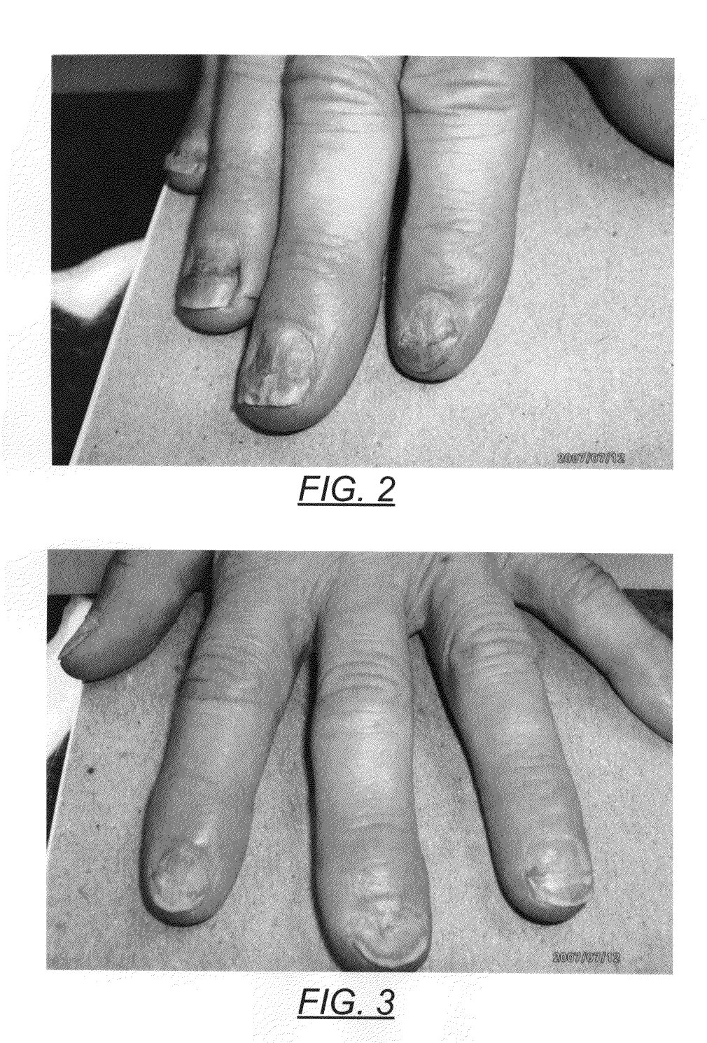

FIG. 2 is a photograph that depicts the right hand after about 8 weeks of treatment.

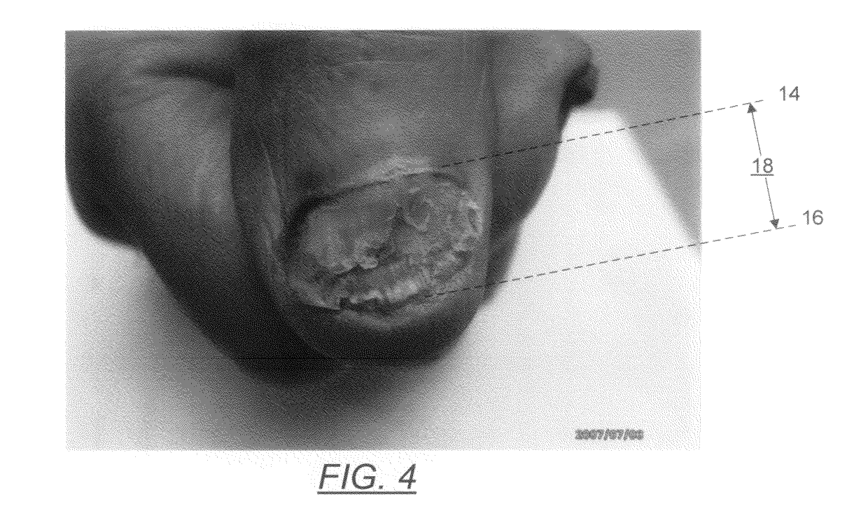

FIG. 3 is a photograph that depicts the left hand after about 8 weeks of treatment.

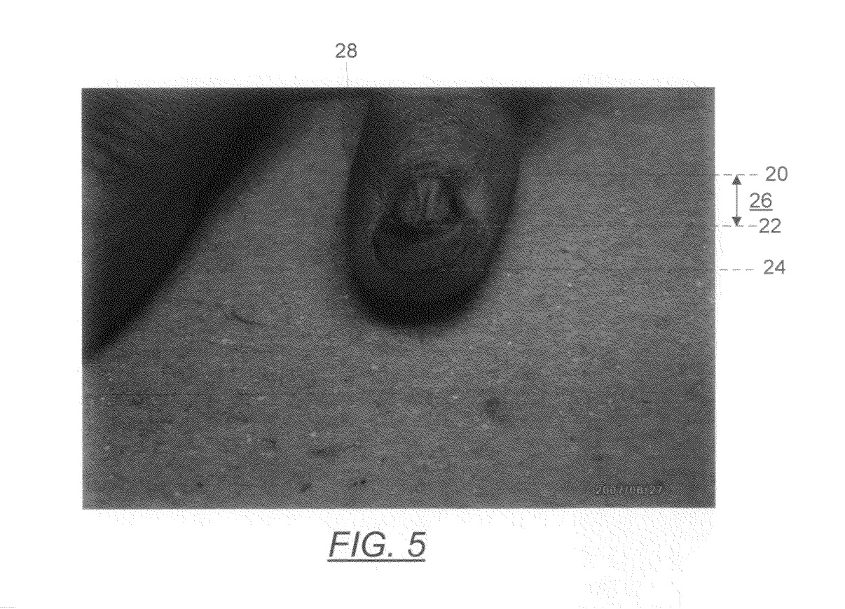

FIG. 4 is a photograph that depicts the thumb of the right hand after about 8 weeks of treatment. The nail growth commenced at 14 and ended at 16, growing a length of 18 during this period.

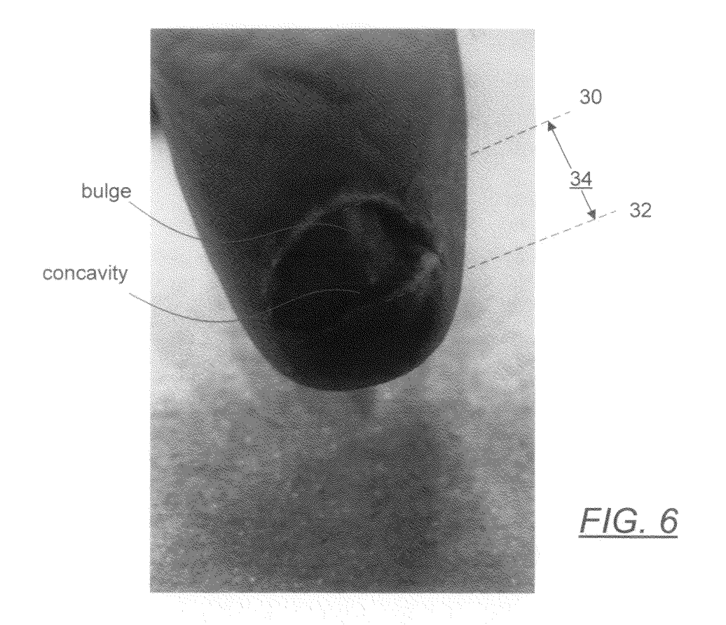

FIG. 5 is a photograph that depicts the index finger of the right hand after about 8 weeks of treatment. The nail growth commenced at 20 and ended at 22 (full nail length ends at 24, including both old nail and new growth portions), growing a length of 26 during this period. The different texture of the fingernails between the new fingernail growth and old fingernail is visually apparent in these photographs. A vertical bulge may be observed at the base of the fingernail.

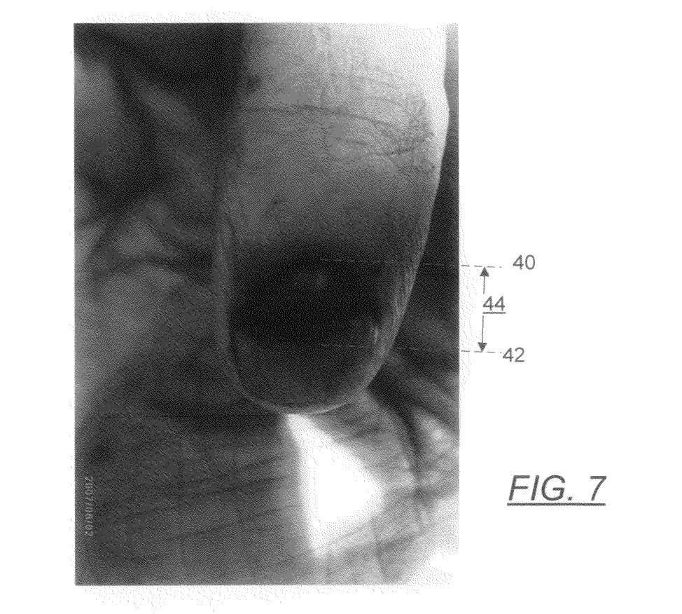

FIG. 6 is a photograph that depicts the index finger of the right hand after about 2 weeks of treatment. The nail growth commenced at 30 and ended at 32, growing a length of 34 during this period. The different texture of the fingernails between the new fingernail growth and old fingernail is visually apparent in these photographs. A bulge and concave portion are identified. Applicant-inventor believes this bulge is indicative of repaid growth of the nail plate.

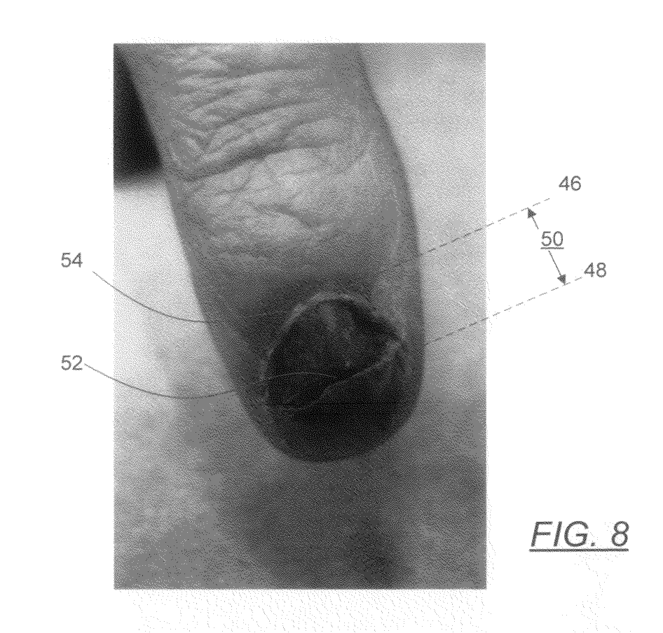

FIG. 7 is a photograph that depicts the ring finger of the right hand after about 2 weeks of treatment. The nail growth commenced at 40 and ended at 42, growing a length of 44 during this period. The different texture of the fingernails between the new fingernail growth and old fingernail is visually apparent in these photographs. The different texture of the fingernails between the new fingernail growth and old fingernail is visually apparent in these photographs.

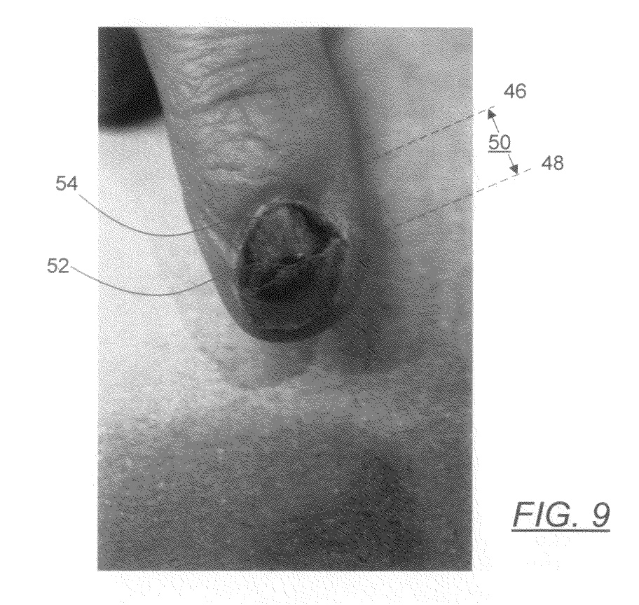

FIGS. 8 and 9 are photographs that depict the middle finger of the right hand after about 2 weeks of treatment. The nail growth commenced at 46 and ended at 48, growing a length of 50 during this period. Applicant-inventor believes this bulge is evidence of the rapid growth of the nail plate. The different texture of the fingernails between the new fingernail growth and old fingernail is visually apparent in these photographs. A bulge 54 and concave portion 52 are identified.

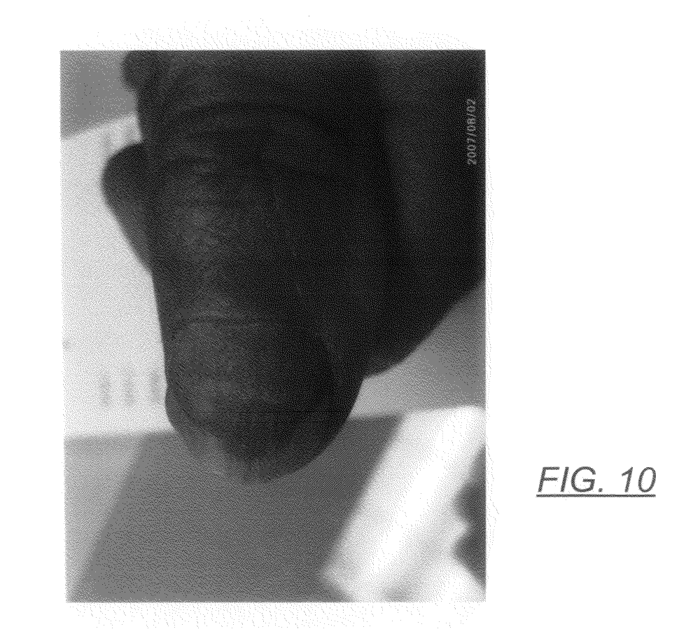

FIG. 10 is a photograph that depicts the thumb of the right hand after treatment. The thumb's fingernail appears normal and healthy following treatment. This photograph was taken about 3 months after the start of the experiment.

Applicant believes that his experimentation has demonstrated that there is a minimum threshold for the concentration of growth factor in the formula below which new cell growth is undetectable. There is also a maximum threshold for growth factor concentration in the formula above which there is no discernible difference in new cell growth and the maximum potential for growth, e.g. the saturation point, has been attained. The optimal concentration for the growth factor that produces health tissue without waste of the growth factor is the combination in Applicant-inventor's formula. After reaching this full potential, the regenerated tissue wall reach a steady state of growth for that tissue, e.g. its homeostasis.

The composition applied to the fingernails was also applied to the scalp and hair. Within two minutes following blending, the composition was topically applied by contacting the scalp and hair with the composition and then allowed to dry at room temperature. It was applied once per day and remained continuously until the hair was washed. After the hair was dried, the composition was reapplied. This was done for 30 continuous days. The remainder of the contents of the blender, the enteral composition, was also consumed once per day during this period. The hair growth (measured by the hair length) demonstrated a rate that was at least 200% the rate prior to administration of the composition. Applicant believes that his experimentation has demonstrated that there is an increase in growth rate. Applicant also believes that hair density will be demonstrably increased with continued use beyond the initial 30 day trial.

### Verification of Utility

Initial verification may be accomplished using traditional laboratory techniques involving double blind studies. One approach would be to use laboratory animals such mice. Mice would be treated with various levels of the active ingredients. Physiological parameters such as mean lifetime, rate of wound healing, pathogenic disease frequency, and organ conditions would be monitored versus untreated animals. Similarly, long term studies could be done with humans, parameters such as sickness frequency, wound healing, blood counts both at healthy state and during illness, physical endurance, and chemical studies to monitor health of various organs, among other test.

In one embodiment, administration of the compound or mixture containing cyanobacteria and a green algae results in the increased production of pluripotent cells from about 1 to about 4 hours following administration. These pluripotent cells will enter the circulatory system, thus increasing the number of circulating pluripotent cells within the subject's body. The percentage increase in the number of circulating pluripotent cells compared to a normal baseline may be about 25%, about 50%, about 100% or greater than about 100% increase as compared to a control. In one embodiment, the control is a base line value from the same subject.

The levels of stem cells available in the circulatory system may be tested according to the teachings of U.S. Pat. No. 6,814,961 disclosing a method to enhance trafficking or homing of stem cells.

As such, those skilled in the art will appreciate that the conception, upon which this disclosure is based, may readily be utilized as a basis for the designing of other structures, methods and systems for carrying out the several purposes of the present invention.

I claim:

1. A composition comprising a therapeutic dosage of green algae and a therapeutic dosage of cyanobacteria, wherein the composition is formulated for topical application.

2. The composition of claim 1, wherein said green algae comprises a green algae selected from the group consisting of Haematococcus pluvialis, Chlorella vulgaris, Chlorella pyrenoidosa, Dunaliella salina or a combination thereof.

3. The composition of claim 1, wherein said cyanobacteria comprises a cyanobacteria selected from the group consisting of Aphanizomenon flos-aquae, Spirulina pacifica, Spirulina plantesis or a combination thereof.

4. The composition of claim 3, wherein said composition comprises Dunaliella salina and Aphanizomenon flos-aquae.

5. The composition of claim 3, wherein said composition comprises Aphanizomenon flos-aquae and Haematococcus pluvialis.

6. The composition of claim 1, wherein said composition further comprises xylitol.

7. The composition of claim 1, wherein the therapeutic dosage comprises from about 30 to about 70 weight percent of said green algae.

8. The composition of claim 1, wherein the therapeutic dosage comprises from about 30 to about 70 weight percent of said cyanobacteria.

9. The composition of claim 1, wherein said composition is comprised in a cosmetic vehicle.

10. The composition of claim 9, wherein said composition comprises a form selected from the group consisting of an emulsion, cream, lotion, solution, anhydrous base, gel, ointment, or combinations thereof.

11. The composition of claim 1, wherein said therapeutic dosage comprises from about 0.01 to about 10.0 weight percent of antioxidants.

12. The composition of claim 11, wherein said therapeutic dosage comprises from about 0.01 to about 6.0 volume percent of astaxanthin.

13. A method for increasing the longevity of epidermal stem cells in a subject, comprising: administering to a subject a topical composition according to claim 1.

14. The method according to claim 13, where said subject is a human.

15. The method according to claim 14, wherein the longevity of epidermal stem cells increased by more than about 4 hours.

16. The method according to claim 15, wherein the composition administered comprises a therapeutic dosage of from about 30 to about 70 weight percent of said cyanobacteria.

17. The method according to claim 16, wherein said cyanobacteria enhances the concentration of epidermal stem cells.

18. The method according to claim 17, wherein said cyanobacteria enhances the concentration of epidermal stem cells by more than about 25%.

19. A method for enhancing a rate of nail growth in a subject, comprising: administering to a subject a topical composition according to claim 1 and an enteral composition comprising a therapeutic dosage of green algae and a therapeutic dosage of cyanobacteria.

20. The method according to claim 19, wherein the rate of nail growth is increased by more than about 200%.

21. A method for increasing a rate of proliferation of epidermal cells in a subject, comprising: administering to a subject a topical composition according to claim 1.

22. The method according to claim 21, wherein the rate of proliferation of epidermal cells is increased by more than about 200%.

23. The method according to claim 21, wherein the method further comprises administration of an enteral composition comprising a therapeutic dosage of green algae and a therapeutic dosage of cyanobacteria.

24. A method for increasing a rate of hair growth in a subject, comprising: administering to a subject a topical composition according to claim 1.

25. The method according to claim 24, wherein the rate of hair growth is increased by more than about 200%.

26. The method according to claim 24, wherein the method further comprises administration of an enteral composition comprising a therapeutic dosage of green algae and a therapeutic dosage of cyanobacteria.

27. A method for increasing a rate of hair follicle stem cell growth in a subject, comprising: administering to a subject a topical composition according to claim 1.

28. The method according to claim 27, wherein the rate of hair follicle stem cell growth is increased by more than about 200%.

29. The method according to claim 27, wherein the method further comprises administration of an enteral composition comprising a therapeutic dosage of green algae and a therapeutic dosage of cyanobacteria.
